(19) United States
(12) Patent Application Publication
(10) Pub. No.: US 2026/0237480 A1
(43) Pub. Date: Aug. 13, 2026

(54) GENERATION AND UPDATE OF TREATMENT PROTOCOLS AND TREATMENT PLANS UTILIZING ARTIFICIAL INTELLIGENCE

(71) Applicant: Align Technology, Inc., San Jose, CA (US)

(72) Inventors: Michael Seeber, Zurich (CH); Evgeniy Malashkin, Madrid (ES); Maksim Surzhikov, Morrisville, NC (US)

(21) Appl. No.: 19/532,956

(22) Filed: Feb. 6, 2026

Related U.S. Application Data
(60) Provisional application No. 63/755,556, filed on Feb. 7, 2025.

(51) Int. Cl. G16H 20/00; G16H 80/00

(52) U.S. Cl. CPC G16H 20/00; G16H 80/00

(57) ABSTRACT

A method includes obtaining first comments indicating changes to a dental treatment plan. The method further includes processing the first comments using one or more AI models. The one or more AI models determine one or more recommended updates to the dental treatment plan that implement the changes from the first comments. The AI models output the one or more recommended updates to the dental treatment plan. The method further includes generating an updated dental treatment plan including the one or more recommended updates, responsive to obtaining user acceptance of the one or more recommended updates.

### RELATED APPLICATIONS

This patent application claims the benefit under 35 U.S.C. § 119(e) of U.S. Provisional Application No. 63/755,556, filed Feb. 7, 2025, which is incorporated by reference herein. This patent application is related to U.S. application Ser. No. 19/448,058, filed Jan. 13, 2026, which is incorporated by reference herein.

### TECHNICAL FIELD

Embodiments of the present invention relate to the field of dentistry, and in particular to the generation and update of treatment protocols and treatment plans by utilizing artificial intelligence models.

### BACKGROUND

When a dentist or orthodontist is engaging with current and/or potential patients, it is often helpful to utilize treatment protocols to assist with treatment planning operations, e.g., a set of operations, preferences, or other instructions that may enable efficient development of a treatment plan for a particular patient or disorder. Further, upon generation of a treatment plan, in some cases updates or changes may be made to further adapt the plan to the particular patient.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the present disclosure, a method includes obtaining first comments indicating changes to a dental treatment plan. The method further includes processing the first comments using one or more artificial intelligence (AI) models. The one or more AI models determine one or more recommended updates to the dental treatment plan. The one or more recommended updates implement changes from the first comments. The one or more AI models output the one or more recommended updates to the dental treatment plan. The method further includes generating an updated dental treatment plan including the one or more recommended updates, responsive to obtaining user acceptance of the one or more recommended updates.

In another aspect of the present disclosure, a method includes obtaining first training data including natural language comments corresponding to target updates of a dental treatment plan. The method further includes obtaining second training data including treatment planning software actions corresponding to the natural language comments. The method further includes training a machine learning model to predict treatment planning software actions based on natural language comments by providing the first training data as training input, and the second training data as target output.

In another aspect of the present disclosure, a method includes obtaining first comments associated with first target updates to a dental treatment plan. The method further includes providing the first comments to a trained machine learning model. The method further includes obtaining, from the trained machine learning model, first output. The first output includes a first updated dental treatment plan. The method further includes providing the first updated dental treatment plan to a computing device associated with a dental treatment provider.

In another aspect of the present disclosure, a method includes obtaining first input including a description of a dental treatment protocol associated with a first target dental disorder. The method further includes providing the first input to a trained machine learning model. The method further includes obtaining, from the trained machine learning model, a first treatment protocol for treating the first target dental disorder in association with the first input in a machine-readable format. The method further includes presenting the first treatment protocol for user approval. The method further includes storing the first treatment protocol in non-transitory memory, responsive to obtaining user approval.

### BRIEF DESCRIPTION OF THE DRAWINGS

The present disclosure is illustrated by way of example, and not by way of limitation in the figures of the accompanying drawings.

FIG. 1 is a block diagram illustrating an exemplary system architecture, according to some embodiments.

FIG. 2 illustrates a model training workflow and a model application workflow, according to some embodiments.

FIG. 3A is a diagram depicting a work flow for generation of a treatment protocol using an AI model, according to some embodiments.

FIG. 3B is a diagram of a graphical user interface (GUI) for use in AI-generated treatment planning operations, according to some embodiments.

FIG. 4A is a flow diagram of a method for generating a dataset for a machine learning model, according to some embodiments.

FIG. 4B is a flow diagram of a method for using one or more AI models to update a dental treatment plan, according to some embodiments.

FIG. 4C is a flow diagram of a method for training a model to perform treatment plan updates based on natural language input, according to some embodiments.

FIG. 4D is a flow diagram of a method for generating treatment plan updates based on output of a trained machine learning model, according to some embodiments.

FIG. 4E is a flow diagram of a method 400E for updating a treatment protocol based on output of a trained machine learning model, according to some embodiments.

FIG. 5A illustrates a tooth repositioning system including a plurality of appliances, in accordance with some embodiments.

FIG. 5B illustrates a method of orthodontic treatment using a plurality of appliances, in accordance with some embodiments.

FIG. 6 illustrates a method for designing an orthodontic appliance to be produced by direct fabrication, in accordance with some embodiments.

FIG. 7 illustrates a method for digitally planning an orthodontic treatment and/or design or fabrication of an appliance, in accordance with some embodiments.

FIG. 8 is a block diagram illustrating a computer system, according to some embodiments.

### DETAILED DESCRIPTION

Described herein are technologies related to improving processes of healthcare services by inclusion and utilization of modeling techniques, including machine learning models, artificial intelligence (AI) models, natural language processing (NLP) models, large language models (LLMs), etc. AI models (e.g., LLMs) may be used to assist in treatment planning, treatment protocol generation, or the like, for various health care services including dental (e.g., orthodontic and/or prosthodontic) health care. AI models may be utilized in translating input from a treatment provider (e.g., a doctor who is not trained in producing machine-readable treatment instructions, three-dimensional modeling related to heath care treatments, or the like) in natural language to a more usable or useful form or format.

A treatment provider may generate one or more protocols for treating health care disorders of interest. For example, a number of common disorders or disorders the practitioner commonly encounters may be candidates for generation of treatment protocols. For example, in the case of orthodontics, a practitioner may have a preference for making some adjustments of dentition before others, preference for aggression of movement, preference for treatment of one type of disorder before another, or the like.

Further, a treatment provider may generate one or more treatment plans, e.g., in association with a particular patient. The treatment plans may be based on treatment protocols, e.g., general preferential guidelines, but may be customized for a specific patient, oral cavity, set of dentition, or the like.

In some systems, generation of a usable protocol or treatment plan may include input by a technician trained to translate a practitioner's protocol notes to a standardized format, such as machine-readable code (e.g., proprietary code, code associated with a particular treatment planning software, or the like). Such systems may require investment in training and paying a number of technicians to support partnered practitioners, maintenance and management of facilities for the technicians, etc. Further, there may be a loss of efficiency in protocol design related to delays between a practitioner requesting an update to a protocol or a new protocol be developed, or generation of a new treatment plan or update to a treatment plan; the technician receiving the practitioner's notes; and generation of the protocol in the updated format. Such delays may be further exacerbated in cases where additional updates to the protocol or treatment plans are requested by the doctor, e.g., including multiple rounds of communication between the technician and practitioner to update or fine-tune the one or more treatment protocols or treatment plans.

Dental arch data may be utilized in treatment of a dental arch. For example, one or more dental malocclusions (e.g., misalignment of teeth) may be treated by an orthodontic treatment plan, which may include collecting and utilizing jaw pair data of a patient. As a further example, generation of a crown or dental implant may be performed based on dental arch data. Dental arch data may include data of one or more teeth (e.g., including size, shape, positioning, orientation, etc.), a group of teeth, an arch, an upper and lower jaw, etc.

A treatment provider, based on examination of a health care disorder (e.g., malocclusion) and/or data collected in connection with the health care disorder (e.g., dental arch data), may design a specific treatment plan with respect to the patient exhibiting the health care disorder. The treatment plan may be related to an existing protocol, be based on an existing protocol, include one or more adjustments from an existing protocol, or the like. In some systems, designing of a treatment plan may include applying a pre-set protocol, determining that one or more portions of the protocol are incorrect, not applied correctly to the specific case, requesting a technician update the protocol, and waiting for the updates to be applied. Similar to initial generation of a protocol, such a process may take multiple steps, multiple rounds of communication, a significant amount of waiting time, etc.

A treatment provider may be unable to parse a treatment plan in a format provided by a technician (e.g., machine-readable code). Review, validation, acceptance, etc., of a treatment plan may further depend on a version of the treatment plan that is translated to a format that may be quickly understood by the practitioner. In some cases, a technician may translate a machine-readable protocol or treatment plan into a natural language or otherwise presented version for review by the practitioner.

Methods and systems of the current disclosure may address one or more shortcomings of conventional solutions. In some embodiments, an AI model is utilized for generation of one or more treatment protocols or treatment plans. The AI model may further be utilized for validation or checking of the treatment protocols or treatment plans. AI models may further be utilized for updating of one or more treatment protocols or treatment plans. The treatment protocols or plans may be related to a variety of healthcare services, including dental, orthodontic, or other services for which creation of a treatment protocol may be of interest.

In some embodiments, a treatment plan may be generated. Generation of the treatment plan may be performed based on a treatment protocol in some embodiments. A description of one or more aspects of the treatment plan, including updates to the treatment plan, may be provided by a treatment provider (e.g., doctor, practitioner, etc.). The description may be provided in natural language. The natural language description may be provided via text input, speech input (e.g., speech-to-text operations, which may be performed by an AI model), etc. The natural language description (e.g., updates to a treatment plan) may be provided to a trained AI model. The trained AI model may be configured to determine, enact, recommend, or the like treatment planning operations in accordance with the natural language comments. In some embodiments, enacting operations based on the practitioner comments may take the form of an “AI copilot,” e.g., a technician using a treatment planning software to enact operations based on practitioner comments may be guided (e.g., via recommended operation) to take actions within the software in accordance with the AI model output related to the practitioner comments. In some embodiments, a technician may instead or additionally make use of an AI copilot function. For example, a technician may provide natural language instructions to adjust some aspects of a treatment plan, such as updating changes made by an AI copilot in response to doctor comments, which may then be enacted within the treatment planning software by the AI copilot in accordance with embodiments of this disclosure. In some embodiments, enacting operations based on practitioner comments may take the form of an “AI technician,” e.g., a treatment plan may be generated based on output of the AI model, which may then be provided for approval, validation, or the like to the practitioner (e.g., without the involvement of a technician trained to operate the treatment planning software, trained to work in a machine-readable coding language associated with treatment planning, or the like).

In some embodiments, a treatment protocol or treatment plan (e.g., a protocol related to a target type of disorder, target treatment type, or the like) may be generated. Generation of the treatment protocol may be performed or assisted by one or more trained AI models. In some embodiments, a treatment provider (e.g., orthodontic practitioner) may provide input in natural language related to treatment protocol for one or more target disorders. The input may be provided by the practitioner via a chat function, a text input, a message (e.g., email), or another input. The input may be provided by the practitioner in natural language, e.g., the practitioner may not be required to undergo special training related to treatment protocol generation.

In some embodiments, a dental treatment plan may be generated by, with, on, or in association with a treatment planning software. For example, a treatment planning software may include functions for defining and/or updating plans for one or more stages of treatment, such as a number of stages corresponding to different appliances in an orthodontic treatment plan. The AI model may be configured to output one or more operations to be performed within the treatment planning software. In some embodiments, the operations within the AI model may include operations that may be performed by a technician, e.g., changes to code, selection of options, input of parameters, or the like that may typically be performed by a technician for updating or creating a treatment plan. Operations of the AI model within the treatment planning software may be based on practitioner comments, additional natural language comments by a technician or another user (e.g., supervising technician), or the like. The updates may be presented as default or recommended actions, which a technician may accept or update, either manually or through the AI copilot, to accelerate the technician's workflow in generating or updating a treatment plan. In some embodiments, the AI model may be configured to output a completed treatment plan, which may or may not include operations performed within a treatment planning software.

In some embodiments, multiple rounds of communication may be used in determining, generating, or updating a treatment plan for treatment of a healthcare disorder (e.g., orthodontic treatment for malocclusion, misalignment, or the like). The multiple communications may include multiple instances of a technician reviewing recommended actions based on output of the AI model, followed by practitioner review of the treatment plan. The multiple communications may include a digital communication, e.g., between an input of the practitioner (e.g., via an AI agent as part of a doctor-facing treatment planning interface) and an AI model, which may result in many updates being completed quickly. In some embodiments, the treatment plan may be used to further generate manufacturing data for one or more appliances to be used during treatment, for initiating manufacturing operations for the one or more appliances, or the like. In some embodiments, the recommended updates may include updates that may be performed by hand, such as adjusting a number of treatment stages in the treatment plan (e.g., a number of orthodontic appliances required to complete treatment), adjusting orthopedic attachments, adjusting an amount of correction for one or more teeth (e.g., introducing over-correction), adjusting torque applied during treatment to one or more teeth, adjusting extrusion or intrusion for one or more teeth, or adjusting a gingiva cut line of an appliance used in treatment.

In some embodiments, treatment plans may include various categories of treatment operations and parameters. Treatment plan parameters may include dental appliance features for one or more dental appliances to be used for the target dental treatment, such as attachments, bite ramps, precision cuts, power ridges, or mandibular advancement features including buccal blocks or occlusal blocks. Attachments may include optimized attachments (such as mesial-distal root control attachments, multi-plane attachments, extrusion attachments, rotation attachments, retention attachments, or expansion support attachments), conventional attachments, or protocol-based attachments (such as G4, G5, G7, or G8 protocol attachments). Treatment plan parameters may also include interproximal reduction (IPR) specifications, including IPR amounts, locations, maximum IPR limits per contact, and staging of IPR operations. Treatment plan parameters may further include staging parameters specifying the number of treatment stages, stage duration, sequencing of movements across stages, and coordination of different treatment operations across the treatment timeline. Treatment plan parameters may include tooth movement parameters such as translation, rotation, torque, tip, intrusion, extrusion, and root movement specifications. Treatment plan parameters may also include overcorrection parameters, passive aligner specifications, midline correction parameters, crossbite correction parameters, crowding resolution parameters, spacing management parameters, and overbite or overjet correction parameters.

In some embodiments, AI models may be provided with information via retrieval-augmented generation (RAG). Documents, clinical guidelines, protocol specifications, and/or other reference materials may be integrated into LLM responses via RAG. In some embodiments, certain responses may be constrained or limited via prompting strategies or RAG. For example, a library of possible machine-readable commands, a library of explanations of various topics, treatments, or disorders, or the like may be provided. The LLM may be constrained to only produce medical explanations that align with provided documentation, only produce machine-readable commands that are included in the library, or the like. RAG techniques may enable the AI model to access up-to-date information without requiring retraining, as the retrieved documentation can be updated independently of the underlying AI model. The retrieved context may be incorporated into the prompt provided to the AI model, enabling the model to generate outputs that are consistent with the retrieved documentation and constrained to valid options defined in the documentation.

For example, in some embodiments, doctor preferences may be stored and retrieved for use in generating treatment plans. A retrieval-augmented generation (RAG) technique may be utilized, where doctor preferences are stored in a data store and provided as context to the AI model when processing treatment instructions. The stored doctor preferences may be generated through various methods, including a questionnaire completed by the dental treatment provider, a chat-based interaction with the dental treatment provider, or automatically based on analysis of historical treatment plan modifications associated with the dental treatment provider. For example, if the AI model observes that a doctor consistently requests certain modifications (e.g., always moving front teeth before back teeth), the system may automatically generate preferences reflecting these patterns. The stored preferences may be retrieved and provided along with doctor comments to the AI model, such that the recommended updates are generated in view of the stored doctor preferences.

In some embodiments, a chat agent may be implemented as a conversational assistant that receives natural language input from the treatment provider and generates contextually appropriate responses, recommendations, or actions based on the input. The chat agent may utilize one or more LLMs as a foundation for understanding and generating natural language. When a user provides input to the chat agent, the input text may be tokenized and processed by the LLM, which generates a probability distribution over possible next tokens or responses based on the input context and the model's learned parameters. The LLM may be configured with specific prompts, system instructions, or fine-tuning that constrain or guide its responses toward treatment planning tasks. The chat agent may maintain session context data comprising a history of prior exchanges between the treatment provider and the chat agent, enabling the LLM to generate responses that account for previous instructions, clarifications, and preferences expressed during the conversation. The chat agent may be configured to perform multiple functions, including answering questions about treatment options or clinical terminology, generating proposed modifications to treatment protocols or treatment plans based on natural language requests, requesting clarification when instructions are ambiguous or incomplete, and providing explanations of proposed changes in human-readable format. The chat agent may be constrained to operate within predefined protocol rules, such that output generated by the underlying LLM is limited to modifications that are valid within a predefined set of protocol commands or treatment options.

In some embodiments, the AI model may be configured to output specific treatment planning software operations or commands. Examples of such operations may include applying three-dimensional (3D) controls to a treatment plan, adjusting staging for one or more teeth, adjusting interproximal reduction (IPR) for one or more teeth, updating a gingiva representation, updating a gingiva cut line (e.g., where an aligner is cut), adding attachments of different types (e.g., optimized attachments, conventional attachments, protocol-based attachments), reducing a number of treatment stages, adding over-correction for specific teeth, adjusting extrusion or intrusion for one or more teeth, changing torque applied to one or more teeth (e.g., molars), and changing a pontic to a semi-pontic at a specified tooth position. These operations may correspond to functions that a technician would typically perform within treatment planning software based on doctor comments.

In some embodiments, even simple modifications requested by a doctor may require multiple operations or clicks within treatment planning software. For example, a request to add over-correction for a specific tooth may involve adding new treatment stages and using 3D controls to implement the over-correction. Fortunately, many treatment planning software functions have keyboard shortcut sequences, which technicians frequently use to accelerate their workflow. In the context of the present disclosure, the AI model may be configured to generate these keyboard shortcut sequences based on the doctor's natural language instructions. In some embodiments, the AI model may generate function calls to an application programming interface (API) of the treatment planning software, enabling direct programmatic control of treatment planning operations.

In some embodiments, two technical implementation approaches may be utilized for AI-assisted treatment planning. A first approach, referred to as a hybrid approach, involves the AI model outputting commands for treatment planning software. In the hybrid approach, the treatment planning software complies with medical regulations, and all possible actions of the AI model are within the approved scope of the treatment planning software. This approach may provide better understandability, as the AI model behaves similarly to a human technician working with the software. The hybrid approach may also be easier to develop, as the action space for the AI model is smaller and constrained to the functionality of the treatment planning software. A second approach, referred to as an end-to-end approach, involves the AI model directly modifying three-dimensional models of patient dentition. The end-to-end approach may be more efficient, as the intermediate treatment planning software is not required. However, the end-to-end approach may be harder to develop and train, as the action space is significantly larger. The end-to-end approach may also raise additional regulatory considerations and may provide less understandability compared to the hybrid approach.

In some embodiments, a domain-specific language (DSL) may be used for expressing treatment protocols in a machine-readable form. The DSL may be a scripting language that expresses doctor preferences in a format that can be automatically processed by treatment planning software. For example, a DSL such as IPL (Invisalign Planning Language) may be used to fully automate doctor protocols within a treatment planning engine. An LLM may be configured to transform natural language input from a treatment provider into DSL scripts expressing the treatment protocol. The DSL scripts may then be used by a treatment engine to automatically generate treatment plans in accordance with the doctor's preferences. In some embodiments, the DSL may support conditional statements, such as specifying different treatment approaches for different malocclusion types or patient characteristics.

In some embodiments, a “smart auto-complete” or copilot functionality may be provided to dental technicians. In this configuration, the AI model provides suggested actions to technicians based on doctor comments, and the technicians may accept the suggestions to accelerate their workflow. The technicians retain the ability to fine-tune the treatment plan using manual controls if the AI suggestions are not fully accurate or if additional adjustments are desired. The technicians may further have the ability to utilize the AI copilot, e.g., may provide natural language instructions to update, correct, or adjust previous changes made by the AI copilot based on doctor comments, to introduce new changes to the treatment planning software which may not have been identified by the AI copilot based on the doctor comments, to improve treatment based on one or more treatment heuristics or best practice parameters, or the like. This copilot approach may serve as a first step in utilizing AI models for treatment planning, enabling collection of training data based on technician acceptance or rejection of AI suggestions. The training data may be used to further improve the AI model's accuracy over time.

In some embodiments, the copilot functionality may enable a technician to engage in an interactive dialogue with the AI model to refine the dental treatment plan. The technician may provide natural language instructions to the AI model in addition to or in response to the doctor's comments. For example, after reviewing the AI model's recommended updates based on the doctor's comments, the technician may provide clarifying instructions, request alternative approaches, or ask questions about the recommended updates. The AI model may respond to the technician's natural language input by generating revised recommendations, providing explanations of the proposed changes, or requesting additional information to resolve ambiguities.

In some embodiments, the technician may engage in a question-and-answer interaction with the AI model via a chat-based interface. The technician may ask the AI model questions such as “why did you recommend this staging approach?” or “what alternatives are available for achieving the requested tooth movement?” The AI model may generate responses that explain the reasoning behind the recommended updates, describe alternative approaches that could achieve similar results, or identify tradeoffs between different treatment options. This interactive dialogue may enable the technician to better understand the AI model's recommendations and make more informed decisions about whether to accept, modify, or reject the suggested updates.

In some embodiments, the technician may provide programmatic instructions to the AI model in addition to or instead of natural language instructions. The programmatic instructions may include specific function calls, parameter values, or sequences of operations to be performed within the treatment planning software. The AI model may interpret the programmatic instructions and generate corresponding updates to the dental treatment plan. In some embodiments, the technician may switch between natural language input and programmatic input based on the complexity of the desired changes or the technician's familiarity with the treatment planning software.

In some embodiments, the standard technician workflow without AI assistance may involve the technician receiving text comments from a doctor, interpreting the doctor's intent from the comments, and manually executing a series of operations within the treatment planning software to implement the requested changes. The manual execution may involve navigating through multiple user interface elements, selecting appropriate tools or functions, entering parameter values, and verifying that each operation produces the intended result. For complex changes requested by the doctor, the technician may need to perform many sequential operations, where each operation may depend on the results of previous operations. The standard workflow may require significant time and expertise, particularly for technicians who must be trained on the specific treatment planning software being used.

In some embodiments, the copilot functionality may improve upon the standard technician workflow by abstracting the complexity of the treatment planning software user interface. Rather than requiring the technician to navigate through multiple menus, dialogs, and tools to implement a requested change, the copilot may present the technician with a suggested sequence of operations that can be accepted with a single action. The technician may review the suggested operations, accept those that correctly implement the doctor's intent, and manually adjust or override those that require modification. This approach may reduce the time required for the technician to process each case while preserving the technician's ability to exercise judgment and make corrections as needed.

In some embodiments, the copilot may be particularly beneficial for changes that require a long chain of operations within the treatment planning software. For example, when a doctor requests a change to the order of tooth movements in a treatment plan, implementing this change may require adjustments to every subsequent stage of treatment. In the standard workflow, the technician may need to manually update each affected stage, which may be time-consuming and error-prone. With the copilot functionality, the AI model may generate the complete sequence of operations needed to implement the requested change across all affected stages, and the technician may accept the entire sequence or selectively modify individual operations as needed.

In some embodiments, the technician may use the copilot to introduce changes that were not explicitly requested by the doctor but that improve the treatment plan based on the technician's expertise or established best practices. For example, the technician may provide natural language instructions to the AI model such as “optimize the staging to minimize treatment duration” or “adjust the attachment placement to improve retention.” The AI model may generate recommended updates based on the technician's instructions, which the technician may then review and accept before the updated treatment plan is provided to the doctor for approval.

In some embodiments, the AI model may handle complex cases requiring long chains of operations. For example, when a doctor requests significant changes to a treatment plan, such as changing the order of tooth movements, every subsequent stage of treatment may need to be adjusted accordingly. A change to one part of the treatment may require cascading updates to subsequent stages. Manually performing such updates may be time-consuming for a technician, but the AI model may significantly speed up this process by generating the full sequence of required operations based on the doctor's high-level instruction. In some embodiments, the one or more recommended updates may comprise a plurality of sequential operations that implement a change specified in the doctor's comments, where the change requires modification of multiple stages of the dental treatment plan.

In some embodiments, the doctor instructions may be provided based on a statement directed toward one or more treatment plans provided by the treatment provider. In some embodiments, an application may be provided to collect doctor comments, e.g., including a graphical user interface. Doctor or practitioner instructions or comments may be provided by one or more GUI input fields. In some embodiments, doctor instructions may be provided via an interactive model. For example, a chat arrangement facilitated by a graphical user interface (GUI) may be utilized in extracting information in natural language from the treatment provider in order to generate one or more treatment protocols. In some embodiments, the first comments may be obtained via a chat-based interface, and the method may further include providing immediate feedback to a user via the chat-based interface based on the one or more recommended updates. In some embodiments, the AI model may predict additional comments based on the first comments, where the first comments are incomplete and the additional comments comprise a completed version of the first comments. The additional comments may be processed together with the first comments to generate the recommended updates, providing a smart auto-complete functionality.

In some embodiments, an AI model may be trained, retrained, adjusted, or the like for one or more operations described. In some embodiments, an AI model may be trained for specific portions of the tasks performed by AI models. For example, an AI model may be specifically trained (adjusted, retrained, or the like) for generating machine-readable instructions based on natural language. Training the AI model may include adjusting parameters of an LLM. Training the AI model may include performing parameter-efficient fine-tuning, low-rank adaptations, adjusting adapter layers, or the like. Training the AI model may be based on one or more test cases, feedback data, feedback evaluation data, indirect feedback data (e.g., based on a number of updates provided to one or more AI-generated protocols), or the like.

In some embodiments, practitioner input for one or more healthcare disorders may be developed into one or more treatment protocols (e.g., outlines for how to treat the applicable disorders in accordance with the practitioner's training and preferences), one or more treatment plans (e.g., application of a protocol to a specific case, patient, or the like), etc. A description of a dental treatment or protocol may be provided to an AI model. From the AI model, a first treatment protocol may be generated (e.g., a machine-readable code outlining one or more treatment protocols associated with one or more dental disorders). The system may, based on machine-readable code indicative of the treatment protocol, generate a form of presentation of the protocol for review by the practitioner. For example, a medical algorithm, which succinctly summarizes steps of the treatment protocol, may be presented via a GUI for approval by the practitioner. The medical algorithm or other approval/validation display may be presented via a deterministic (e.g., not based on AI or machine learning) model. The system, upon obtaining user approval based on the review, may proceed with storing the first treatment protocol.

In some embodiments, further validation may be performed by applying the treatment protocol to one or more test cases, e.g., examples of the target disorder, one or more prior patients or others for which disorder data is available, or the like. A result of a treatment may be presented via a GUI for validation by the practitioner. Further updates may be made based on natural language input and AI model output if the final results are not acceptable to the practitioner.

Aspects of the present disclosure provide technological advantages compared to conventional methods. Aspects of the present disclosure may enable generation of treatment protocols and treatment plans without and/or while reducing the role of a technician. Such methods may reduce reliance on a technician, reduce turnaround time for treatment protocol or plan updates or generation, reduce additional delays that may be caused by a technician being in a different time zone or having a different working schedule than a practitioner, or the like. Such methods further improve efficiency by reducing a requirement of training for technicians in parsing doctor comments and generating machine-readable instructions related to protocol generation. In some embodiments, a practitioner could generate one or more treatment protocols or plans essentially immediately without the involvement of any additional personnel.

Aspects of the present disclosure further improve operations of practitioner review of proposed treatment plans and treatment protocols, e.g., via a GUI including validation operations. The GUI may present a medical algorithm or another recognizable format for doctor review. The GUI may present predicted final conditions (e.g., after treatment) for the current case, for one or more test cases (e.g., in the case of a treatment protocol), or the like. The final conditions (e.g., models or images indicative of final tooth positions for an orthodontic treatment) may be used by a practitioner to approve generation or updates to one or more treatment plans or protocols.

In one aspect of the present disclosure, a method includes obtaining first comments indicating changes to a dental treatment plan. The method further includes processing the first comments using one or more AI models. The one or more AI models determine one or more recommended updates to the dental treatment plan that implement the changes from the first comments. The AI models output the one or more recommended updates to the dental treatment plan. The method further includes generating an updated dental treatment plan including the one or more recommended updates, responsive to obtaining user acceptance (e.g., via practitioner review) of the one or more recommended updates.

In another aspect of the present disclosure, a method includes obtaining first training data including natural language comments corresponding to target updates of a dental treatment plan. The method further includes obtaining second training data. The second training data includes treatment planning software actions corresponding to the natural language comments. The method further includes training a machine learning model to predict treatment planning software actions based on natural language comments by providing the first training data as training input and the second training data as target output.

In another aspect of the present disclosure, a method includes obtaining first comments associated with first target updates to a dental treatment plan. The method further includes providing the first comments to a trained machine learning model. The method further includes obtaining output including a first updated dental treatment plan from the trained machine learning model. The method further includes providing the first updated dental treatment plan to a computing device associated with a dental treatment provider.

In another aspect of the present disclosure, a method includes obtaining first input including a description of a dental treatment protocol associated with a first target dental disorder. The method further includes providing the first input to a trained machine learning model. The method further includes obtaining a first treatment protocol for treating the first target dental disorder in association with the first input. The first treatment protocol is obtained from the trained machine learning model. The first treatment protocol is expressed in a machine-readable format. The method further includes presenting the first treatment protocol for user approval. The method further includes storing the first treatment protocol in non-transitory memory, responsive to obtaining user approval.

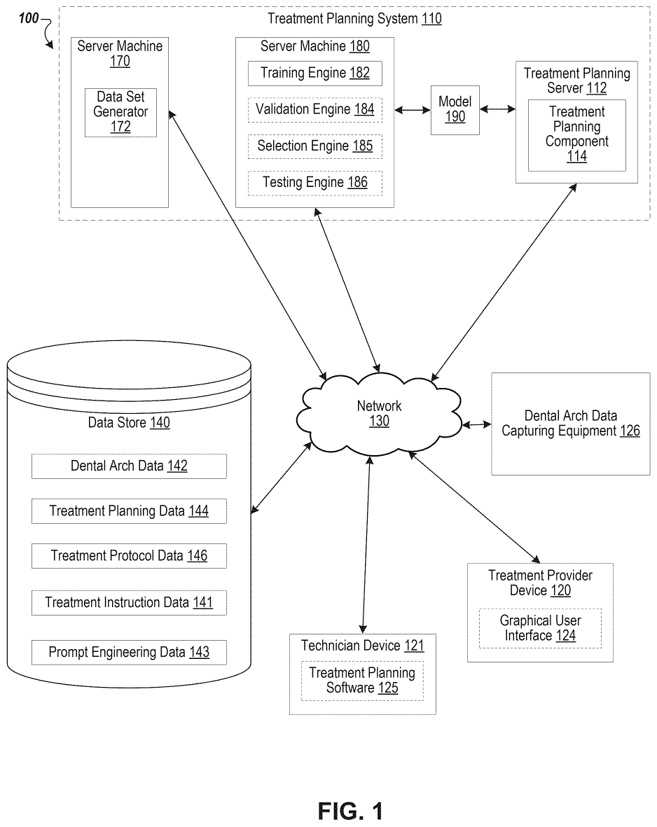

FIG. 1 is a block diagram illustrating an exemplary system 100 (exemplary system architecture), according to some embodiments. The system 100 includes a treatment provider device 120, dental arch data capturing equipment 126, treatment planning server 112, technician device 121, and data store 140. The treatment planning server 112 may be part of treatment planning system 110. Treatment planning system 110 may further include server machines 170 and 180. As used herein, when appropriate, techniques, operations, or components related to dental arches and dental arch data may be extended to include operations related to other oral structures, including upper and/or lower gingiva and palate. For example, dental arch data capturing equipment 126 may further be used for performing intraoral scans including generating data of other oral structures in addition to teeth.

Dental arch data capturing equipment 126 may include any combination of equipment for collecting dental arch data, examples of which include intraoral scanners, x-ray machines, cameras, and so on. In some embodiments, dental arch data capturing equipment 126 corresponds to an intraoral scanner as described in U.S. Publication No. 2019/0388193, filed Jun. 19, 2019, entitled “Intraoral 3D Scanner Employing Multiple Miniature Cameras and Multiple Miniature Pattern Projectors,” which is incorporated by reference herein. In some embodiments, dental arch data capturing equipment 126 corresponds to an intraoral scanner as described in U.S. application Ser. No. 16/910,042, filed Jun. 23, 2020 and entitled “Intraoral 3D Scanner Employing Multiple Miniature Cameras and Multiple Miniature Pattern Projectors,” which is incorporated by reference herein. In some embodiments, dental arch data capturing equipment 126 corresponds to an intraoral scanner as described in U.S. Pat. No. 10,835,128, issued Nov. 17, 2020, which is incorporated by reference herein. In some embodiments, dental arch data capturing equipment 126 corresponds to an intraoral scanner as described in U.S. Pat. No. 10,918,286, issued Feb. 21, 2021, which is incorporated by reference herein. Dental arch data may include data of healthy dental arches, dental arches including malocclusion or teeth misalignment, etc.

In some embodiments, treatment provider device 120 may be used to generate or collect doctor instructions related to one or more treatment protocols or treatment plans, which may be included in treatment instruction data 141 of data store 140. Treatment instruction data 141 may store doctor comments, notes, or instructions related to treatment protocols or treatment plans. Treatment instruction data 141 may relate to natural language input by one or more doctors, healthcare professionals, treatment providers, practitioners, etc. Treatment instruction data 141 may be provided via graphical user interface 124 of treatment provider device 120. Treatment instruction data 141 may relate to one or more treatment protocols, e.g., general plans meant to relate to one or more generic, common, repeatedly encountered, or the like healthcare disorders (e.g., dental disorders, malocclusion, misalignment, or the like). Treatment instruction data 141 may relate to specific cases, e.g., treatment plans for a particular patient.

Doctor instructions may be processed (e.g., by the treatment provider device 120 and/or by the treatment planning server 112). Processing of the treatment instruction data 141 may include generating features. In some embodiments, the features are a pattern in the treatment instruction data 141 (e.g., relationships between input words, phrases, tokens, etc.) or a combination of values from the treatment instruction data 141. Generating features may include tokenizing the treatment instruction data 141. Treatment instruction data 141 may include features and the features may be used by treatment planning component 114 for performing signal processing and/or for obtaining treatment protocol data 146 and/or treatment planning data 144, e.g., for generation of healthcare treatments. In some embodiments, features may include segmentation data of treatment instruction data 141. Segmentation may be performed (e.g., using a trained machine learning model trained to perform instance segmentation or semantic segmentation), and may include separation of various portions of doctor input into different sections, portions, or categories, such as sections related to different disorders, treatment types, or the like. Each instance (e.g., set) of treatment instruction data 141 may correspond to an individual (e.g., practitioner), a group of similar dental arches, or the like.

In some embodiments, treatment planning system 110 may generate treatment protocol data 146 and/or treatment planning data 144 using supervised machine learning. In some embodiments, treatment planning system 110 may generate treatment protocol data 146 and/or treatment planning data 144 based on providing doctor instructions to one or more instances of a natural language processing model, an LLM, or the like. In some embodiments, treatment protocol data 146 and/or treatment planning data 144 may be or include machine readable instructions, e.g., related to target positions of one or more teeth to treat orthodontic disorders, related to treatment operations or steps for treating dental disorders, or the like. In some embodiments, treatment planning system 110 may generate treatment protocol data 146 and/or treatment planning data 144 using unsupervised machine learning (e.g., treatment protocol data 146 and/or treatment planning data 144 includes output from a machine learning model that was trained using unlabeled data, output may include clustering results, principal component analysis, anomaly detection, etc.). In some embodiments, treatment planning system 110 may generate treatment protocol data 146 and/or treatment planning data 144 using semi-supervised learning (e.g., training data may include a mix of labeled and unlabeled data, etc.).

In some embodiments, generation of treatment protocol data 146 and/or treatment planning data 144 (e.g., by providing treatment instruction data 141 to an LLM) may also include providing further data to an AI model, such as instructions to process the input in a target way. Such instructions may be stored as prompt engineering data 143. Prompt engineering data 143 may include one or more prompts, prompt forms, prompt generation algorithms, or the like for generating accompanying data to be provided along with data associated with doctor input to an AI model. Prompt engineering data 143 may include additional text to be provided to an LLM that may be inserted before, in the middle of, after, or otherwise with a selection of doctor input or data based on doctor input (e.g., pre-processed or processed doctor input). Prompt engineering data 143 may cause an LLM to perform a different task based on which prompt is provided, e.g., one LLM may be used for many different tasks by providing input data accompanied with various additional prompt information. In some embodiments, an LLM may be specifically trained to perform a target task (such as generating machine-readable treatment protocol instructions based on natural language input), and prompt engineering data 143 may not be used.

Treatment provider device 120, dental arch data capturing equipment 126, treatment planning server 112, data store 140, server machine 170, technician device 121, and server machine 180 may be coupled to each other via network 130 for generating treatment protocol data 146 and/or treatment planning data 144, e.g., to generate treatment protocol machine-readable instructions based on natural language practitioner input. In some embodiments, network 130 may provide access to cloud-based services. Operations performed by treatment provider device 120, treatment planning system 110, data store 140, etc., may be performed by virtual cloud-based devices.

In some embodiments, network 130 is a public network that provides treatment provider device 120 with access to the treatment planning server 112, data store 140, technician device 121, and other publicly available computing devices. In some embodiments, network 130 is a private network that provides treatment provider device 120 access to dental arch data capturing equipment 126, data store 140, and other privately available computing devices. Network 130 may include one or more Wide Area Networks (WANs), Local Area Networks (LANs), wired networks (e.g., Ethernet network), wireless networks (e.g., an 802.11 network or a Wi-Fi network), cellular networks (e.g., a Long Term Evolution (LTE) network), routers, hubs, switches, server computers, cloud computing networks, and/or a combination thereof.

Treatment provider device 120 and/or technician device 121 may include computing devices such as Personal Computers (PCs), laptops, mobile phones, smart phones, tablet computers, netbook computers, network connected televisions (“smart TV”), network-connected media players (e.g., Blu-ray player), a set-top-box, Over-the-Top (OTT) streaming devices, operator boxes, etc. Treatment provider device 120 may include a graphical user interface 124. Graphical user interface 124 may receive user input (e.g., via a Graphical User Interface (GUI) displayed via the treatment provider device 120) of an indication associated with dental arch data. In some embodiments, treatment provider device 120 transmits the indication to the treatment planning system 110, receives output (e.g., treatment protocol data 146 and/or treatment planning data 144) from the treatment planning system 110, determines a treatment protocol or update, and causes the protocol to be displayed to the treatment provider via graphical user interface 124.

Corrective actions may be associated with design of a treatment protocol, updating of a treatment protocol, providing an alert associated with a treatment protocol to a user, or the like.

Treatment planning server 112, server machine 170, and server machine 180 may each include one or more computing devices such as a rackmount server, a router computer, a server computer, a personal computer, a mainframe computer, a laptop computer, a tablet computer, a desktop computer, Graphics Processing Unit (GPU), accelerator Application-Specific Integrated Circuit (ASIC) (e.g., Tensor Processing Unit (TPU)), etc. Operations of treatment planning server 112, server machine 170, server machine 180, data store 140, etc., may be performed by a cloud computing service, cloud data storage service, etc.

Treatment planning server 112 may include a treatment planning component 114. In some embodiments, the treatment planning component 114 may receive treatment instruction data 141, (e.g., receive from the treatment provider device 120, retrieve from the data store 140) and generate output (e.g., treatment protocol data 146 and/or treatment planning data 144) based on the input data. In some embodiments, treatment protocol data 146 and/or treatment planning data 144 may include machine-readable instructions (e.g., computer code, code related to a treatment planning software, or the like) for one or more healthcare disorders. In some embodiments, treatment protocol data 146 and/or treatment planning data 144 may include indications of changes to be made within treatment planning software 125, e.g., in the case of an AI assistant embodiment of the present disclosure. In some embodiments, such as a case of an AI technician replacing a human technician, operations of technician device 121 may be performed by treatment provider device 120 or another device, or not performed at all, as appropriate.

System 100 may include one or more machine leaning or AI models, e.g., model 190. AI models may perform many tasks, including mapping dental arch data to a latent space, splitting an input up into related sections, formatting input text, detecting subject matter, transforming natural language instructions into machine-readable instructions, performing clinical checking, or the like. Model 190 may be trained using dental instruction data. Model 190 may be a general purpose LLM, configured via prompt engineering to perform one or more tasks based on treatment provider input.

One type of machine learning model that may be used to perform some or all of the above tasks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs).

A recurrent neural network (RNN) is another type of machine learning model. A recurrent neural network model is designed to interpret a series of inputs where inputs are intrinsically related to one another, e.g., time trace data, sequential data, etc. Output of a perceptron of an RNN is fed back into the perceptron as input, to generate the next output.

A graph convolutional network (GCN) is a type of machine learning model that is designed to operate on graph-structured data. Graph data includes nodes and edges connecting various nodes. GCNs extend CNNs to be applicable to graph-structured data which captures relationships between various data points. GCNs may be particularly applicable to meshes, such as three-dimensional data.

Many other types and varieties of machine learning models may be utilized for one or more embodiments of the present disclosure. Further types of machine learning models that may be utilized for one or more aspects include transformer-based architectures, generative adversarial networks, volumetric CNNs, etc. Selection of a specific type of machine learning model may be performed responsive to an intended input and/or output data, such as selecting a model adapted to three-dimensional data to perform operations on three-dimensional models of dental arches, a model adapted to two-dimensional image data to perform operations based on images of a patient's teeth, etc.

Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

A large language model (LLM) is a type of AI model designed to understand and generate human-like text, natural language, or the like. LLMs are generally built using deep learning techniques and trained on large datasets from diverse sources. LLMs often provide natural language understanding, text generation, contextual learning, instruction following based on nuanced or detailed prompts, and other functions. LLMs have advantages based on a large extent of knowledge mapped by the layers of the models, as well as an ability to make correct connections between concepts to generate relevant output. For many operations described in this disclosure, it may be convenient to utilize one or more large language models. However, different language models, including natural language processing (NLP) models, small language models, or specialized language models may be utilized in place of one or more LLMs described in operations herein. For example, an operation described as including multiple LLMs may include one large language model, one natural language processing model, one small language model, and one specialized language model specifically tuned for a target use case (such as a dental treatment case) and still be within the scope of this disclosure. Language processing models can vary greatly in scope, power, usage requirements, training requirements, etc., and a different combination of model properties may be utilized to target various tasks of interest with respect to healthcare treatment planning, dental treatments, orthodontic treatments, etc. In particular, the most generally powerful large language models are often trained on any and all publicly available data, and include many tunable parameters (e.g., on the order of tens of billions to trillions of tunable parameters). Small language models are often more focused, including fewer tunable parameters (e.g., millions or billions of parameters) and may be trained using a subset of publicly available data, such as data from verified sources or data associated with a target field of interest. Specialized language models may be trained using domain specific data, including private data, and may be or include adjustments to general small language models.

In some embodiments, treatment planning component 114 receives doctor protocol descriptions in natural language, performs signal processing to break down the current data into sets of current data (e.g., via segmentation or paragraph splitting, which may be provided by an LLM such as model 190), provides the sets of current data as input to a trained model 190, and obtains outputs indicative of treatment protocol data 146 and/or treatment planning data 144 from the trained model 190. In some embodiments, model 190 may represent a single general purpose or specifically trained or adjusted LLM, which may be utilized multiple times using different engineered prompts to perform various tasks in association with a set of doctor protocol description, instructions, input, etc.

In some embodiments, the various models discussed in connection with model 190 (e.g., supervised machine learning model, unsupervised machine learning model, etc.) may be combined in one model (e.g., a hierarchical model), or may be separate models.

Data may be passed back and forth between several distinct models included in model 190 and treatment planning component 114, or provided to a single model multiple times. In some embodiments, some or all of these operations may instead be performed by a different device, e.g., treatment provider device 120, technician device 121, server machine 170, server machine 180, etc. It will be understood by one of ordinary skill in the art that variations in data flow, which components perform which processes, which models are provided with which data, and the like are within the scope of this disclosure.

Data store 140 may be a memory (e.g., random access memory), a drive (e.g., a hard drive, a flash drive), a database system, a cloud-accessible memory system, or another type of component or device capable of storing data. Data store 140 may include multiple storage components (e.g., multiple drives or multiple databases) that may span multiple computing devices (e.g., multiple server computers). The data store 140 may store treatment instruction data 141, treatment protocol data 146 and/or treatment planning data 144, prompt engineering data 143, and dental arch data 142. Dental arch data may include data obtained by dental arch data capturing equipment 126. Dental arch data 142 may be used by system 100 to test or validate one or more treatment plans or treatment protocols, e.g., a protocol may be applied to a test case or a previous patient case included in dental arch data 142, and the results evaluated by a treatment provider to determine whether the treatment protocol is accurate, is working as intended, is acceptable, or the like.

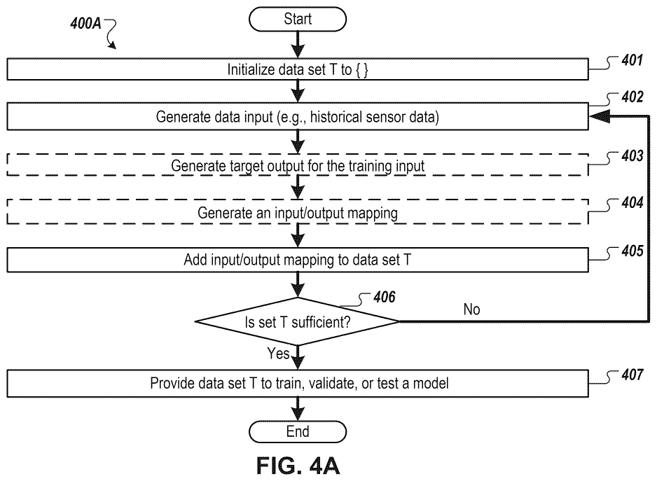

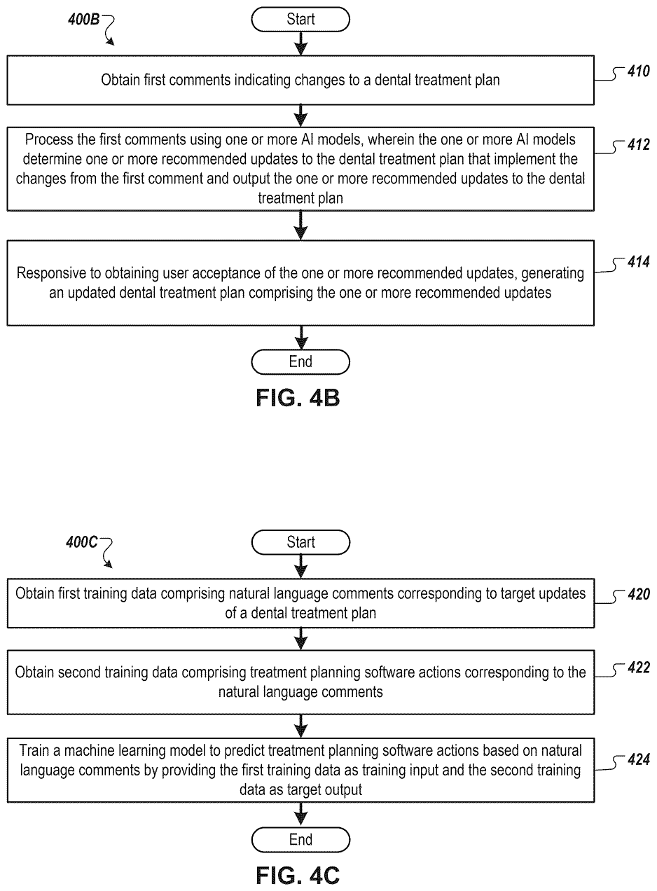

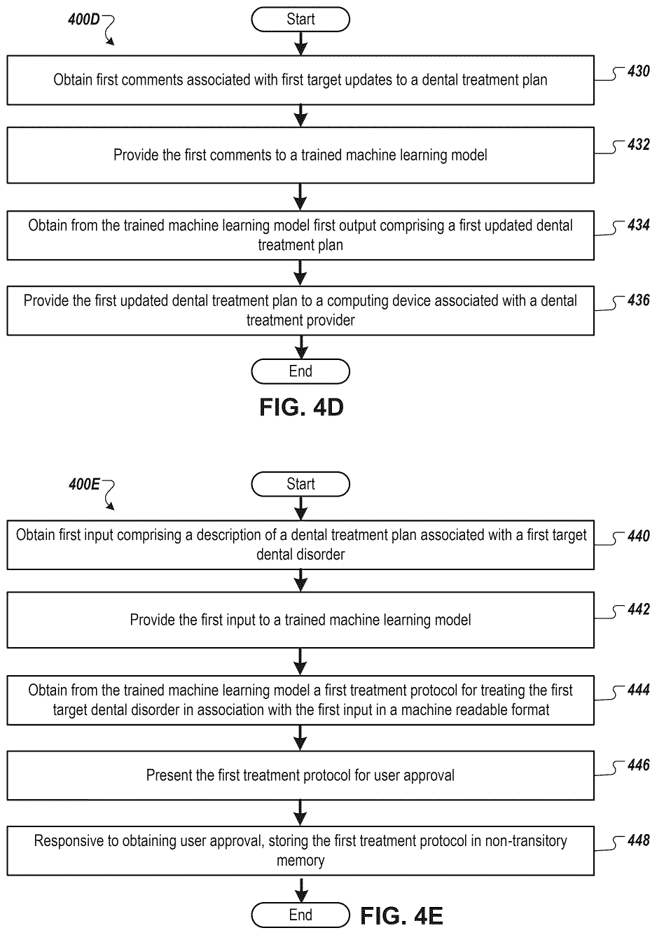

In some embodiments, treatment planning system 110 further includes server machine 170 and server machine 180. Server machine 170 includes a data set generator 172 that is capable of generating data sets (e.g., a set of data inputs and a set of target outputs) to train, validate, and/or test model(s) 190, including one or more machine learning models. Some operations of data set generator 172 are described in detail below with respect to FIG. 4A. In some embodiments, data set generator 172 may partition the historical data (e.g., historical instruction data) into a training set (e.g., sixty percent of the historical data), a validating set (e.g., twenty percent of the historical data), and a testing set (e.g., twenty percent of the historical data).

In some embodiments, treatment planning system 110 (e.g., via treatment planning component 114) generates multiple sets of features based on processing of input data. For example a first set of features may correspond to a first subset of instruction data (e.g., data related to a first disorder, first type of treatment, or the like) that correspond to each of the data sets (e.g., training set, validation set, and testing set) and a second set of features may correspond to a second subset of instruction data that correspond to each of the data sets.

In some embodiments, machine learning model 190 is provided historical data as training data. Training data may be used to adjust one or more parameters of an existing model, e.g., to retrain or focus the model for use in a particular application. Various types of retraining schemes may be used, that may be more efficient than fully retraining an AI model, LLM, or the like. The type of data provided will vary depending on the intended use of the machine learning model. For example, a machine learning model may be trained by providing the model with historical doctor inputs as training input. The machine learning model 190 may be configured to generate machine-readable instructions related to doctor instructions. A machine learning model may be provided with corresponding machine-readable instructions, e.g., in a target programming language, instructions related to actions that could be taken by a technician with respect to treatment planning software 125, or the like. Such a machine learning model may be configured to generate mappings (e.g., in a latent space) between natural language instructions and machine-readable instructions.

In one embodiment, server machine 180 includes a training engine 182, a validation engine 184, selection engine 185, and/or a testing engine 186. An engine (e.g., training engine 182, a validation engine 184, selection engine 185, and a testing engine 186) may refer to hardware (e.g., circuitry, dedicated logic, programmable logic, microcode, processing device, etc.), software (such as instructions run on a processing device, a general purpose computer system, or a dedicated machine), firmware, microcode, or a combination thereof. The training engine 182 may be capable of training a model 190 using one or more sets of features associated with the training set from data set generator 172. The training engine 182 may generate multiple trained models 190, where each trained model 190 corresponds to a distinct set of features of the training set (e.g., instruction data from a specific subset of treatment categories, such as disorder types, treatment types, disorder severities, or another categorization). For example, a first trained model may have been trained using all features (e.g., X1-X5), a second trained model may have been trained using a first subset of the features (e.g., X1, X2, X4), and a third trained model may have been trained using a second subset of the features (e.g., X1, X3, X4, and X5) that may partially overlap the first subset of features. Data set generator 172 may receive the output of a trained model (e.g., segmented input data into paragraphs each related with a target topic), collect that data into training, validation, and testing data sets, and use the data sets to train a second model (e.g., a machine learning model configured to perform further operations based on the segmented input data, etc.).

Validation engine 184 may be capable of validating a trained model 190 using a corresponding set of features of the validation set from data set generator 172. For example, a first trained machine learning model 190 that was trained using a first set of features of the training set may be validated using the first set of features of the validation set. The validation engine 184 may determine an accuracy of each of the trained models 190 based on the corresponding sets of features of the validation set. Validation engine 184 may discard trained models 190 that have an accuracy that does not meet a threshold accuracy. In some embodiments, selection engine 185 may be capable of selecting one or more trained models 190 that have an accuracy that meets a threshold accuracy. In some embodiments, selection engine 185 may be capable of selecting the trained model 190 that has the highest accuracy of the trained models 190.

Testing engine 186 may be capable of testing a trained model 190 using a corresponding set of features of a testing set from data set generator 172. For example, a first trained machine learning model 190 that was trained using a first set of features of the training set may be tested using the first set of features of the testing set. Testing engine 186 may determine a trained model 190 that has the highest accuracy of all of the trained models based on the testing sets.

In the case of a machine learning model, model 190 may refer to the model artifact that is created by training engine 182 using a training set that includes data inputs and corresponding target outputs (correct answers for respective training inputs). Patterns in the data sets can be found that map the data input to the target output (the correct answer), and machine learning model 190 is provided mappings that capture these patterns. The machine learning model 190 may use one or more of Support Vector Machine (SVM), Radial Basis Function (RBF), clustering, supervised machine learning, semi-supervised machine learning, unsupervised machine learning, k-Nearest Neighbor algorithm (k-NN), linear regression, random forest, neural network (e.g., artificial neural network, recurrent neural network, CNN, graph neural network, GCN), etc.

Treatment planning component 114 may provide current data to model 190 and may run model 190 on the input to obtain one or more outputs. For example, treatment planning component 114 may provide target treatment instruction data 141 to model 190 and may run model 190 on the input to obtain one or more outputs. Treatment planning component 114 may be capable of determining (e.g., extracting) treatment protocol data 146 and/or treatment planning data 144 from the output of model 190. Treatment planning component 114 may determine (e.g., extract) confidence data from the output that indicates a level of confidence that predictive data (e.g., treatment protocol data 146 and/or treatment planning data 144) is an accurate representation of the natural language instructions provided by the treatment provider. Treatment planning component 114 may use the confidence data to decide whether to cause a corrective action associated with the dental arch, e.g., providing a prompt to the practitioner to provide additional clarity, additional information, or the like.

The confidence data may include or indicate a level of confidence that the treatment protocol data 146 and/or treatment planning data 144 is an accurate prediction associated with the input data. In one example, the level of confidence is a real number between 0 and 1 inclusive, where 0 indicates no confidence that the treatment protocol data 146 and/or treatment planning data 144 is an accurate prediction for the input data and 1 indicates absolute confidence that the treatment protocol data 146 and/or treatment planning data 144 accurately predicts machine-readable instructions associated with the input data. Responsive to the confidence data indicating a level of confidence below a threshold level for a predetermined number of instances (e.g., percentage of instances, frequency of instances, total number of instances, etc.) treatment planning component 114 may cause trained model 190 to be re-trained (e.g., based on an updated pool of training data, etc.). In some embodiments, retraining may include generating one or more data sets (e.g., via data set generator 172) utilizing historical data.

For purpose of illustration, rather than limitation, aspects of the disclosure describe the training of one or more machine learning models 190 using historical data and inputting current data into the one or more trained machine learning models to determine treatment protocol data 146 and/or treatment planning data 144. In other embodiments, a heuristic model, physics-based model, or rule-based model is used to determine treatment protocol data 146 and/or treatment planning data 144 (e.g., without or in addition to using a trained machine learning model. Any of the information described with respect to data inputs to one or more models may be monitored or otherwise used in the heuristic, physics-based, or rule-based model.

In some embodiments, the functions of treatment provider device 120, treatment planning server 112, server machine 170, and/or server machine 180 may be provided by a fewer number of machines. For example, in some embodiments server machines 170 and 180 may be integrated into a single machine, while in some other embodiments, server machine 170, server machine 180, and treatment planning server 112 may be integrated into a single machine. In some embodiments, treatment provider device 120 and treatment planning server 112 may be integrated into a single machine. In some embodiments, functions of treatment provider device 120, treatment planning server 112, server machine 170, server machine 180, and data store 140 may be performed by a cloud-based service.

In general, functions described in one embodiment as being performed by treatment provider device 120, treatment planning server 112, server machine 170, and server machine 180 can also be performed on treatment planning server 112 in other embodiments, if appropriate. In addition, the functionality attributed to a particular component can be performed by different or multiple components operating together. For example, in some embodiments, the treatment planning server 112 may determine a corrective action based on the treatment protocol data 146 and/or treatment planning data 144. In another example, treatment provider device 120 may determine the treatment protocol data 146 and/or treatment planning data 144 based on output from the trained machine learning model.

In addition, the functions of a particular component can be performed by different or multiple components operating together. One or more of the treatment planning server 112, server machine 170, or server machine 180 may be accessed as a service provided to other systems or devices through appropriate application programming interfaces (API).

In embodiments, a “user” may be represented as a single individual. However, other embodiments of the disclosure encompass a “user” being an entity controlled by a plurality of users and/or an automated source. For example, a set of individual users federated as a group of administrators may be considered a “user.”

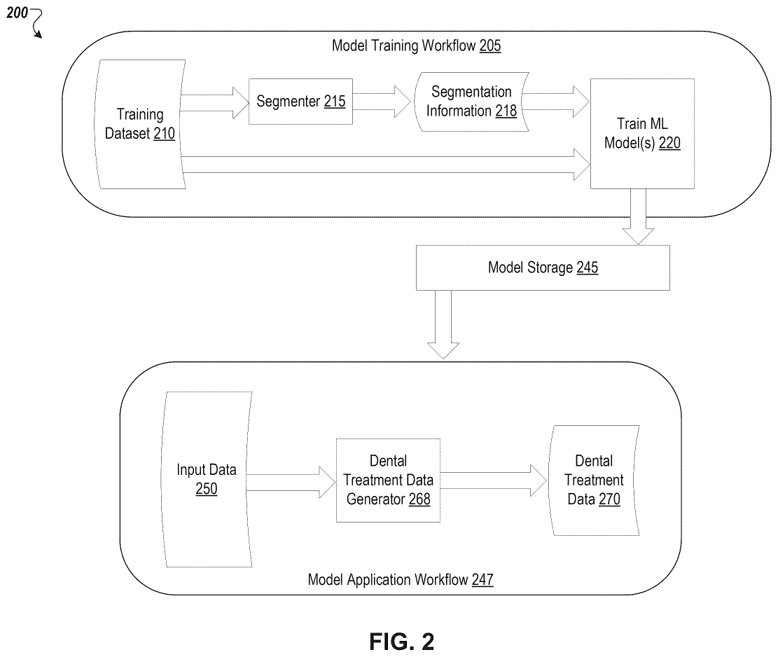

FIG. 2 illustrates workflows 200 for training and implementing one or more machine learning models for performing operations associated with utilizing practitioner instructions to generate treatment protocols and/or treatment plans, in accordance with embodiments of the present invention. The illustrated workflows include a model training workflow 205 and a model application workflow 247. The model training workflow 205 is to train or retrain one or more machine learning models (e.g., deep learning models, generative models, LLMS, etc.) to perform one or more data segmentation tasks and/or data generation tasks. The model application workflow 247 is to apply the one or more trained machine learning models to generate treatment protocol data, for instance in the form of machine-readable instructions for protocol generation based on disorder details, based on the input data 250.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

The model training workflow 205 and the model application workflow 247 may be performed by processing logic, executed by a processor of a computing device. Workflows 205 and 247 may be implemented, for example, by one or more devices depicted in FIG. 1, such as server machine 170, server machine 180, treatment planning server 112, etc. These methods and/or operations may be implemented by one or more machine learning modules executed on processing devices of devices depicted in FIG. 1.

For the model training workflow 205, a training dataset 210 containing hundreds, thousands, tens of thousands, hundreds of thousands or more examples of input data may be provided. The properties of the input data will correspond to the intended use of the machine learning model(s). For example, a machine learning model for processing natural language instructions to produce machine readable instructions for generating treatment plans may be trained. Training the machine learning model for generating machine readable protocol instructions may include providing a training dataset 210 of natural language input data to be mapped to output machine readable instructions. In some embodiments, the natural language input data may be provided as output by one or more additional AI models, e.g., via language translation, speech-to-text operations, or the like. The output machine readable instructions may be in a programming language used for generating treatment plans, may be indicative of actions to be taken in a treatment planning software, or the like. Training dataset 210 may include additional information, such as contextual information, metadata, etc.

In some embodiments, workflows 200 may be associated with fine-tuning or adjusting a general-purpose artificial intelligence or machine learning model, e.g., for adjusting operations of an LLM to cause the LLM to be more applicable to the target operations of generating treatment protocol instructions based on natural language instruction input. One or more operations as described with respect to model training workflow 205 and/or model application workflow 247 may be performed in a generalized context, e.g., without particular emphasis on input data related to treatment protocol generation. Such a general-purpose AI model (e.g., an LLM) may produce satisfactory results when utilized to generate protocol data in accordance with the present disclosure. In some embodiments, one or more techniques utilizing a training dataset 201 specific to target operations of the model may be used to adjust a generally trained model to perform target tasks with a higher degree of accuracy.

Techniques for adjusting a general-purpose LLM may include parameter-efficient fine-tuning operations. Parameter-efficient fine-tuning is a technique to update AI models in a resource-efficient manner. Parameter-efficient fine-tuning may reduce computational and data volume demands by only updating a subset of a models parameters, e.g., according to methods described with respect to model training workflow 205. Parameter-efficient fine-tuning may include adapter layer tuning, which includes introducing small neural network layers or adapters between existing layers of a pre-trained model. During fine-tuning, only the adapter layers are trained. Parameter-efficient fine-tuning may include lor-rank adaptation techniques, which add low-rank matrices to the weight matrices of the model. During fine-tuning, only these low-rank matrices are updated. Parameter-efficient fine-tuning methods may include bias tuning, where only bias terms are adjusted, prefix-tuning, where learnable vectors are added to input embeddings that are adjusted during fine-tuning, techniques where some parameters are allowed to be updated while others are held static, or other methods for improving performance of a pre-trained AI model.

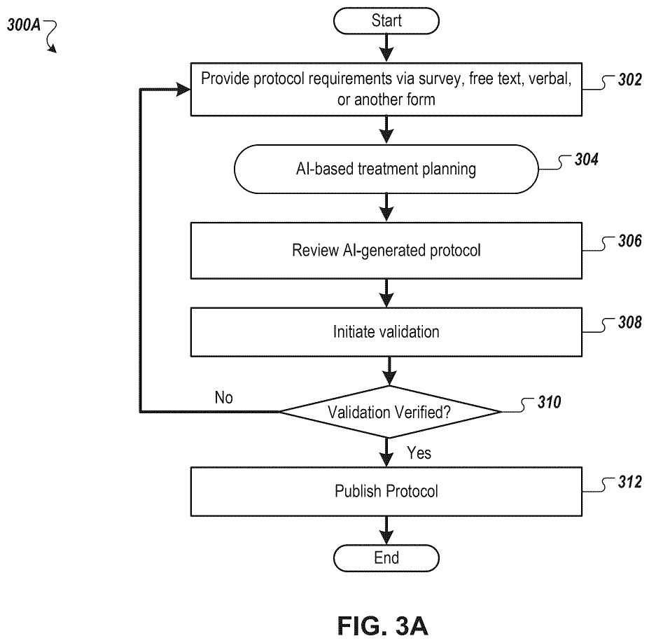

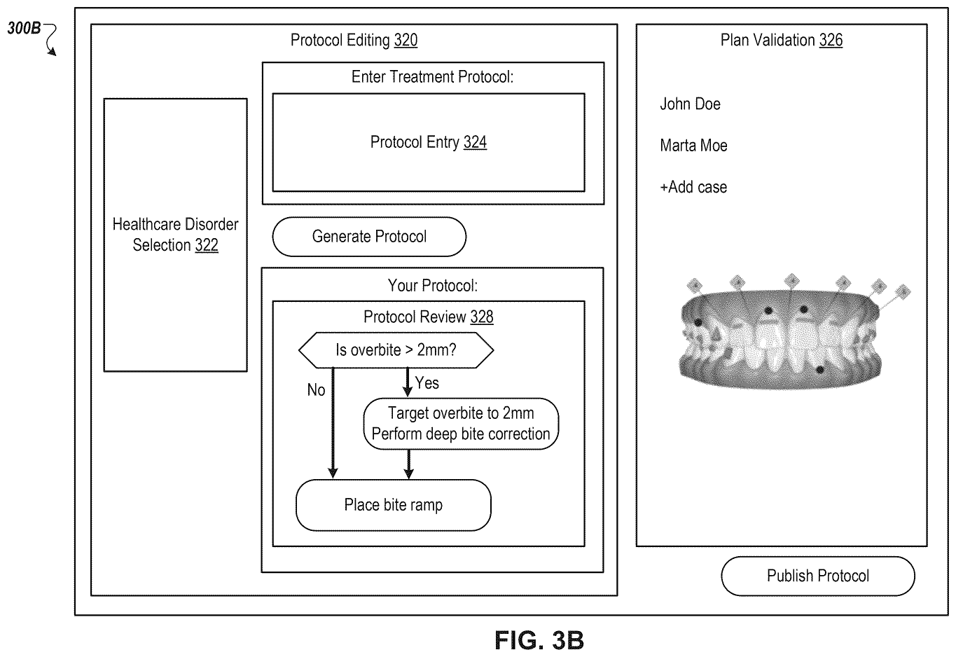

Training dataset 210 may reflect the intended use of the machine learning model. A model may be configured to generate machine-readable protocol instructions. For example, a model may be configured to translate natural language protocol descriptions into machine-readable protocol instructions, or one or more formats or types of instructions for updating operations of a treatment planning software (e.g., indications that cause the treatment planning software to default, auto-fill, or recommend particular changes, updates or actions for a treatment plan). The machine learning model configured to predict protocol instructions may be provided with data indicative of one or more natural language and machine-readable instruction pairs as part of training dataset 210. Such a model may be trained to receive a natural language description of a treatment protocol, including practitioner generate treatment guidelines or preferences for a particular healthcare disorder, and generate as output machine-readable instructions that may facilitate generating a specific treatment plan based on disorders of a target patient. In some embodiments, an LLM may be trained (e.g., adjusted, retrained) to perform one or more different tasks with respect to generating machine-readable instructions, such as tasks described with respect to FIG. 3D.

In some embodiments, some or all of the training dataset 210 may be segmented. For example, a model may be trained or configured (e.g., via training adjustments or prompt engineering) to separate input related to multiple topics into sections, each associated with one of the input topics. For example, an input may include descriptions of treatment preferences or protocols related to several disorders, and a segmenter may be used to separate the input into sections related to each target disorder. The segmenter 215 may separate portions of data for training of a machine learning model. For example, individual topics, such as separate disorders, like may be segmented from each other for generating data sets for training a model to generate treatment protocol data.

Data of the training dataset 210 may be processed by segmenter 215 that segments the data of training dataset 210 into multiple different features. The segmenter may then output segmentation information 218. The segmenter 215 may itself be a machine learning model, e.g., a machine learning model configured to separate input into different sections, segments, categories, or the like. Segmenter 215 may be a general purpose LLM, may be an LLM trained specifically to be applicable to dental disorders, an LLM trained specifically to segment natural language instructions related to dental treatment protocols, or the like. In some embodiments, training dataset 210 may not be provided to segmenter 215, e.g., training dataset 210 may be provided to train ML models without segmentation.

In some embodiments, various other pre-processing operations (e.g., in addition to or instead of segmentation) may also be performed before providing input (e.g., training input or inference input) to the machine learning model. Other pre-processing operations may share one or more features with segmenter 215 and/or segmentation information 218, e.g., location in the model training workflow 205. Pre-processing operations may include mesh closing, artifact removal, various text formatters, or other pre-processing that may improve performance of the machine learning models.

Data from training dataset 210 may be provided to train one or more machine learning models at block 220. Training a machine learning model may include first initializing the machine learning model. The machine learning model that is initialized may be a deep learning model such as an artificial neural network. An optimization algorithm, such as back propagation and gradient descent may be utilized in determining parameters of the machine learning model based on processing of data from training dataset 210.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many inputs in the training dataset yields a network that can produce correct output when presented with inputs that are different than the ones present in the training dataset. In high-dimensional settings, such as large images, this generalization is achieved when a sufficiently large and diverse training dataset is made available.

An artificial neural network includes an input layer that consists of values in a data point (e.g., tokenizing may occur to map each input word or phrase to a token). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer.

Processing logic adjusts weights of one or more nodes in the machine learning model(s) based on an error term. The error term may be based upon a difference between output of the machine learning model and target output provided as part of training dataset 210. For example, the error term may be based upon a difference between output of the machine learning model based on a natural language input, and machine-readable instructions associated with the natural language input (e.g., provided by a subject matter expert). Based on this error, the artificial neural networks adjust one or more of their parameters for one or more of their nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

In some embodiments, portions of available training data (e.g., training dataset 210) may be utilized for different operations associated with generating a usable machine learning model. Portions of training dataset 210 may be separated for performing different operations associated with generating a trained machine learning model. Portions of training dataset 210 may be separated for use in training, validating, and testing of machine learning models. For example, 60% of training dataset 210 may be utilized for training, 20% may be utilized for validating, and 20% may be utilized for testing.

In some embodiments, the machine learning model may be trained based on the training portion of training dataset 210. Training the machine learning model may include determining values of one or more parameters as described above to enable a desired output related to an input provided to the model. One or more machine learning models may be trained, e.g., based on different portions of the training data. The machine learning models may then be validated, using the validating portion of the training dataset 210. Validation may include providing data of the validation set to the trained machine learning models and determining an accuracy of the models based on the validation set. Machine learning models that do not meet a target accuracy may be discarded. In some embodiments, only one machine learning model with the highest validation accuracy may be retained, or a target number of machine learning models may be retained. Machine learning models retained through validation may further be tested using the testing portion of training dataset 210. Machine learning models that provide a target level of accuracy in training operations may be retained and utilized for future operations. At any point (e.g., validation, testing), if the number of models that satisfy a target accuracy condition does not satisfy a target number of models, training may be performed again to generate more models for validation and testing.

Once one or more trained machine learning models are generated, they may be stored in model storage 245, and utilized for generating predictive data associated with dental treatment protocols, such as providing predictions of machine-readable instructions associated with natural language protocol or plan descriptions or updates, operations related to a flow to generate the instructions, or the like.

In some embodiments, model application workflow 247 includes utilizing the one or more machine learning models trained at block 220. Machine learning models may be implemented as separate machine learning models or a single combined (e.g., hierarchical, ensemble model, or the like) machine learning model in embodiments.

Processing logic that applies model application workflow 247 may further execute a user interface, such as a graphical user interface. A user may select one or more options using the user interface. Options may include selecting which of the trained machine learning models to use, selecting which of the operations the trained machine learning models are configured to perform to execute, customizing input and/or output of the machine learning models, or the like. For example, a user may only be interested in output of a single or a set of disorders, but may provide doctor-generated natural language input related to a number of disorders or other input that is not relevant to the target output. In some embodiments, a GUI may provide multiple elements associated with entry of different comments for different treatment plans, treatment protocols, or the like.

Input data 250 is provided to a machine learning model trained in block 220. The input data 250 may correspond to at least a portion or training dataset 210, e.g., be the same type of data, data that resembles data of training dataset 210, or the like. Input data 250 may include new natural language instructions for treatment protocols, e.g., from a new practitioner, a practitioner who wishes to update their standard treatment protocols, etc. Input data 250 may further include ancillary information, metadata, labeling data, etc.

In some embodiments, input data may be preprocessed. For example, preprocessing operations performed on the training dataset 210 may be repeated for at least a portion of input data 250. Input data 250 may include segmented data, data with anomalies or outliers removed, data with manipulated mesh data, or the like.

Input data is provided to dental treatment data generator 268. Dental treatment data generator 268 generates dental treatment plan data 270 (e.g., treatment protocol data 146 and/or treatment planning data 144 of FIG. 1) based on the input data 250. In some embodiments, dental treatment data generator 268 includes a single trained machine learning model. In some embodiments, dental treatment data generator 268 includes a combination of multiple trained machine learning models. In some embodiments, dental treatment data generator 268 includes multiple applications of input provided to a single trained AI model, e.g., an LLM may be provided various prompts including doctor input to perform multiple operations to facilitate generation of dental treatment plan data 270. In some embodiments, dental treatment data generator 268 may include a combination of machine learning models and other models. For example, a combination of machine learning models and numerical optimization models may be included in dental treatment data generator 268. In some embodiments, one or more sets of data may be generated based on protocol data, such as data for generating a treatment plan, data for generating manufacturing plans for one or more appliances for treatment, or the like. In some embodiments, a corrective action may be performed based on the dental treatment plan data 270. The corrective action may include providing an alert to a user, designing a treatment plan, updating a treatment plan, or the like.

FIG. 3A is a diagram depicting a work flow 300A for generation of a treatment protocol using an AI model, according to some embodiments. Similar operations could also be performed for use in generating a specific treatment plan. Generation of a treatment protocol may be based on one or more statements or instructions provided in natural language by a treatment provider, practitioner, doctor, or the like. Work flow 300A depicts actions to be performed by a practitioner (e.g., doctor, treatment provider) in association with a system such as system 100 of FIG. 1 for the generation of a treatment protocol utilizing one or more AI models. Some operations may include input of a technician, e.g., in an AI copilot embodiment.

Flow 300A begins at block 302 with a practitioner providing protocol requirements. The protocol requirements may be provided via a GUI, e.g., executed by a computing device associated with the practitioner. The protocol requirements may be provided in a variety of ways and/or formats. For example, a survey style input may be collected via a GUI, a free form text input may be acquired (e.g., via email or another messaging platform), voice input may be acquired (which may require preprocessing to generate text input, which may be performed by AI, in some embodiments), or the like.

At block 304, the input is provided to an AI-based treatment planning platform. The AI-based treatment platform may be or include an LLM, e.g., the AI-based treatment planning platform may include an AI agent that is configured to communicate with (provide input to and obtain output from) an LLM. In some embodiments, the AI model(s) may be executed on the same device by which the practitioner inputs protocol requirements (e.g., treatment provider device 120 of FIG. 1). In some embodiments, the AI models may be executed on a different device (e.g., technician device 121 of FIG. 1).

In some embodiments (e.g., in the case of an AI copilot implementation), the AI-based treatment planning operations may include updating one or more settings, selections, or the like of a treatment planning software. One or more iterations of AI-based and/or manual treatment planning may occur at a technician device. For example, a technician may be presented with AI-based selections in a treatment planning software, which were determined based on natural language comments by a practitioner. The technician may accept or further update the treatment planning software to be in alignment with the perceived intentions of the doctor, e.g., based on the natural language comments. This AI-generated protocol which has been reviewed and/or edited by a technician may be provided for practitioner review at block 306.

At block 306, the AI-generated protocol is reviewed by the practitioner. The protocol (or treatment plan) may be presented in a format that is easily understood by the practitioner, e.g., not in a format that requires special training, such as a programming or machine-readable code, a set of operations in a treatment planning software, or the like. In some embodiments, a description of the protocol in natural language may be provided. In some embodiments, output of the AI model (e.g., machine-readable output) may be provided to a medical algorithm generator, and the medical algorithm generator may be used to generate a protocol for review. Generation of the protocol in a reviewable format may be deterministic or rule-based, e.g., not performed by an AI model.

At block 308, validation operations are initiated. Validation operations may include further opportunities for a treatment provider to review a treatment protocol. Validation operations may enable a treatment provider to observe results of applying the treatment protocol to a healthcare or dental disorder. For example, validation operations may include applying a treatment protocol to one or more test cases stored in memory. In the case of orthodontic treatment, example dentition, and/or dentition of one or more previous patients, may be used to validate the treatment protocol. Results of applying the protocol may be presented to the treatment provider, e.g., in the form of a model or image of the teeth after treatment has been performed. The treatment provider may verify that the treatment protocol has had the desired effect.

At block 310, flow 300A splits based on whether the validation operations confirmed the efficacy of the AI-generated protocol. If the AI-generated protocol is not performing as intended, flow returns to block 302, and further input including a restatement of the practitioners intentions, requested updates to the protocol, or the like may be provided. If the AI-generated protocol is performing target operations, the protocol is published in block 312 for future use in treating patients.

FIG. 3B is a diagram of GUI 300B for use in AI-generated treatment planning operations, according to some embodiments. GUI 300B may be executed by a function or application of a computing device belonging to or associated with a practitioner or treatment provider. GUI 300B may provide a method for the treatment provider to access one or more AI models for use in developing treatment protocols. GUI 300B may be part of a treatment planning software. GUI 300B may be associated with an application that acts as an AI agent, that performs actions including providing input to and/or receiving input from an AI model, that provides input to another device or application that acts as an AI agent, or the like. In some embodiments, GUI 300B may be associated with an application that provides input of the treatment provider to a technician device, which acts as an AI agent.

GUI 300B includes protocol editing 320 and plan validation 326. Protocol editing may be associated with providing natural language comments to an AI model for generation of a treatment protocol. Plan validation 326 may include applying the generated protocol to one or more test cases to ensure or validate that the protocol is acting as intended by the treatment provider.

Protocol editing 320 may include healthcare disorder selection 322. GUI 300B may be used in generating treatment protocols for one or more of a set of applicable disorders. A particular healthcare disorder may be selected, and other elements of GUI 300B may be directed toward that disorder. In this way, protocols for a number of different disorders (e.g., a number of different dental or orthodontic disorders, such as deep bite, crowding, premolar extraction or the like) may be generated, edited, reviewed, published, etc. using GUI 300B.

Upon optional selection of a healthcare disorder, protocol entry 324 may be utilized for providing treatment provider comments related to the protocol. Natural language comments may be entered to be provided to one or more AI models for generation of a treatment protocol. The natural language comments may describe treatment preferences, sequencing of treatment operations, parameters for specific treatment steps, conditions under which certain treatment approaches should be applied, or other aspects of the treatment protocol that the treatment provider wishes to specify. In some embodiments, the protocol entry 324 may include a text input field, a chat-based interface, or other input mechanisms that enable the treatment provider to express treatment protocol requirements in natural language without requiring specialized training in machine-readable code or treatment planning software operations. In some embodiments, additional information may be provided to the AI or LLM, e.g., prompt engineering configuring the AI model to perform the target task. The prompt engineering data may include instructions that guide the AI model to interpret the natural language comments in the context of dental treatment planning, constrain the AI model output to valid treatment operations, or configure the AI model to generate output in a specific machine-readable format suitable for subsequent processing by treatment planning software or treatment engines.

A treatment protocol may be generated based on the natural language entry. For protocol review 328, GUI 300B may provide an overview of the treatment protocol based on protocol entry 324 in a manner that may be understood by the treatment provider. In some embodiments, a medical algorithm such as that shown in FIG. 3B may be generated that indicates details of the treatment protocol. As illustrated in FIG. 3B, protocol review 328 may display a flowchart-style medical algorithm that visually represents the decision points and treatment steps of the generated protocol, enabling the treatment provider to quickly verify the logical flow and sequencing of treatment operations. The illustrated medical algorithm may include branching decision nodes, treatment action nodes, and connecting arrows that indicate the progression of treatment based on various patient conditions or treatment outcomes. Other formats for protocol review 328 may include a natural language description of the protocol, a tabular summary listing treatment steps with associated parameters and conditions, a hierarchical outline format organizing treatment phases and sub-steps, a timeline-based visualization showing the temporal sequence of treatment operations, or a comparison view displaying the generated protocol alongside a reference or default protocol to highlight customizations. In some embodiments, protocol review 328 may present multiple format options simultaneously or allow the treatment provider to toggle between different presentation formats based on preference. In some embodiments, generation of the protocol review 328 may be deterministic, e.g., may not be performed by an AI model. For example, machine readable code may be provided as input to a protocol review model, and the protocol review model may generate a medical algorithm or other format for review by the treatment provider. Any differences between intended treatment and the generated treatment may be perceived by the treatment provider at this stage, and updated instructions may be provided via protocol entry 324 for further review, refinement, update, etc.

Plan validation 326 may provide further opportunity for a practitioner to verify that a treatment protocol is presented as intended. Plan validation 326 may include a selection of test cases (e.g., fictional dentition exhibiting a target disorder, previous patient dental data, or the like). The AI-generated treatment protocol may be applied to one or more test cases. In some embodiments, a 3D model of the upper and/or lower dental arches indicative of a result of treatment may be provided for the treatment provider to verify. The 3D model may be presented via an interactive viewer within the graphical user interface that enables the treatment provider to manipulate the view of the resultant dentition. The treatment provider may pan the 3D model to shift the viewing position horizontally or vertically, enabling inspection of different regions of the dental arches. The treatment provider may rotate the 3D model about one or more axes to view the dentition from different angles, such as buccal, lingual, occlusal, or oblique perspectives. The treatment provider may zoom in on the 3D model to inspect fine details of individual teeth, interproximal contacts, or occlusal relationships, and may zoom out to view the overall arch form and alignment. In some embodiments, the interactive viewer may provide preset viewing angles corresponding to standard clinical views, such as frontal, left lateral, right lateral, and occlusal views. The treatment provider may also toggle between views of the upper arch, lower arch, or both arches in occlusion to evaluate the bite relationship. In some embodiments, the interactive viewer may enable the treatment provider to compare the initial dentition arrangement with the predicted final arrangement by toggling between views or displaying the arrangements side-by-side. For example, final dentition positions may be demonstrated via an image or three-dimensional model that are predicted to result from applying the AI-generated protocol to one or more test cases. Upon verifying that the protocol is performing as intended, the treatment provider may publish the protocol for use in other cases, for future patients, or the like.

FIGS. 4A-E are flow diagrams of methods 400A-E associated with training and utilizing models for generating treatment protocols, according to certain embodiments. Methods 400A-E may be performed by processing logic that may include hardware (e.g., circuitry, dedicated logic, programmable logic, microcode, processing device, etc.), software (such as instructions run on a processing device, a general purpose computer system, or a dedicated machine), firmware, microcode, or a combination thereof. In some embodiment, methods 400A-E may be performed, in part, by treatment planning system 110 of FIG. 1. Method 400A may be performed, in part, by treatment planning system 110 (e.g., server machine 170 and data set generator 172 of FIG. 1). Treatment planning system 110 may use method 400A to generate a data set to at least one of train, validate, or test a machine learning model, in accordance with embodiments of the disclosure. Methods 400B-E may be performed by treatment planning server 112 (e.g., treatment planning component 114), treatment provider device 120, and/or server machine 180 (e.g., training, validating, and testing operations may be performed by server machine 180). In some embodiments, a non-transitory machine-readable storage medium stores instructions that when executed by a processing device (e.g., of treatment planning system 110, of server machine 180, of treatment planning server 112, etc.) cause the processing device to perform one or more of methods 400A-E.

For simplicity of explanation, methods 400A-E are depicted and described as a series of operations. However, operations in accordance with this disclosure can occur in various orders and/or concurrently and with other operations not presented and described herein. Furthermore, not all illustrated operations may be performed to implement methods 400A-E in accordance with the disclosed subject matter. In addition, those skilled in the art will understand and appreciate that methods 400A-E could alternatively be represented as a series of interrelated states via a state diagram or events.

FIG. 4A is a flow diagram of a method 400A for generating a data set for a machine learning model, according to some embodiments. Referring to FIG. 4A, in some embodiments, at block 401 the processing logic implementing method 400A initializes a training set T to an empty set.

At block 402, processing logic generates first data input (e.g., first training input, first validating input). The first data input may include data types related to an intended use of the machine learning model. The first data input may include natural language instructions related to a treatment of a dental disorder. The first data input may include data appropriate for other operations, such as data of several topics to be split into sections, statements to be converted to machine-readable instructions, statements to be detected as default statements, or other data characteristics related to generation of AI-based treatment protocols.

In some embodiments, at block 403, processing logic optionally generates a first target output for one or more of the data inputs (e.g., first data input). In some embodiments, target output may represent an intended output space for the model. For example, a machine learning model configured to generate machine-readable instructions may be provided natural language instructions and machine-readable instructions as training input and corresponding target output. A machine learning or AI model configured to output actions in a treatment planning software may receive natural language description as training input and corresponding actions in a treatment planning software as target output.

At block 404, processing logic optionally generates mapping data that is indicative of an input/output mapping. The input/output mapping (or mapping data) may refer to the data input (e.g., one or more of the data inputs described herein), the target output for the data input, and an association between the data input(s) and the target output. In some embodiments, data segmentation may also be performed. For example, various sections related to different disorders or treatments may be separated for further operations. In some embodiments, such as in association with machine learning models where no target output is provided, block 404 may not be executed.

At block 405, processing logic adds the mapping data generated at block 404 to data set T, in some embodiments.

At block 406, processing logic branches based on whether data set T is sufficient for at least one of training, validating, and/or testing a machine learning model, such as model 190 of FIG. 1. If so, execution proceeds to block 407, otherwise, execution continues back at block 402. It should be noted that in some embodiments, the sufficiency of data set T may be determined based simply on the number of inputs, mapped in some embodiments to outputs, in the data set, while in some other embodiments, the sufficiency of data set T may be determined based on one or more other criteria (e.g., a measure of diversity of the data examples, accuracy, etc.) in addition to, or instead of, the number of inputs.

At block 407, processing logic provides data set T (e.g., to server machine 180) to train, validate, and/or test machine learning model 190. In some embodiments, data set T is a training set and is provided to training engine 182 of server machine 180 to perform the training. In some embodiments, data set T is a validation set and is provided to validation engine 184 of server machine 180 to perform the validating. In some embodiments, data set T is a testing set and is provided to testing engine 186 of server machine 180 to perform the testing. In the case of a neural network, for example, input values of a given input/output mapping (e.g., numerical values associated with data inputs) are input to the neural network, and output values (e.g., numerical values associated with target outputs) of the input/output mapping are stored in the output nodes of the neural network. The connection weights in the neural network are then adjusted in accordance with a learning algorithm (e.g., back propagation, etc.), and the procedure is repeated for the other input/output mappings in data set T. After block 407, a model (e.g., model 190) can be at least one of trained using training engine 182 of server machine 180, validated using validating engine 184 of server machine 180, or tested using testing engine 186 of server machine 180. The trained model may be implemented by treatment planning component 114 (of treatment planning server 112) to generate treatment protocol data 146 and/or treatment planning data 144 for performing signal processing, or for performing a corrective action.

FIG. 4B is a flow diagram of a method 400B for using one or more AI models to update a dental treatment plan, according to some embodiments. Actions similar to those of method 400B may be used for other types of healthcare disorders, e.g., other than dental disorders. At block 410, process logic obtains first comments indicating changes to a dental treatment plan. The first comments may be or include natural language input. The first comments may be provided by a practitioner, doctor, treatment provider, etc. The first comments may be provided via text input, chat function, email, or the like. The first comments may be provided via voice processing, e.g., by an AI model. In some embodiments the first comments may be or include a correction or update to a dental treatment plan, e.g., updates to a plan generated based on second comments. The dental treatment plan may be or include an orthodontic treatment plan to correct orthodontic disorders, such as malocclusion or tooth misalignment.

At block 412, process logic processes the first comments using one or more AI models. The one or more AI models may determine one or more recommended updates to the dental treatment plan. The one or more recommended updates may implement the changes from the first comments and output the one or more recommended updates to the dental treatment plan. The recommended updates may include adjustments associated with actions to be performed in a treatment planning software, updates or additions to machine-readable instructions, or the like.

In embodiments, the recommended updates are recommendations for portions of or aspects to a dental treatment plan. The recommended updates may be implementations of the first comments and/or may include further treatment suggestions in view of the first comments. For example, the first comments may specify that a target number of stages are desired, and the recommended updates may indicate proposed final treatment outcomes that are achievable in the specified number of treatment stages.

In some embodiments, the first output comprising the first updated dental treatment plan may include one or more recommended updates generated by the trained machine learning model based on the first comments. The recommended updates may include reducing a number of stages included in the updated dental treatment plan, such as by consolidating tooth movements across fewer treatment stages to shorten overall treatment duration. The recommended updates may include adjusting orthopedic attachments, such as modifying the type, size, position, or configuration of attachments to be placed on one or more teeth during treatment. The recommended updates may include adding over-correction for one or more teeth, where the treatment plan specifies tooth movements beyond the target final position to account for expected relapse or settling after treatment completion. The recommended updates may include adjusting torque applied during treatment to one or more teeth, such as modifying the labial or lingual root torque for anterior teeth or buccal or lingual root torque for posterior teeth. The recommended updates may include adjusting extrusion or intrusion of one or more teeth, where extrusion moves a tooth in an occlusal direction and intrusion moves a tooth in an apical direction. The recommended updates may include adjusting a gingiva cut line of an appliance used in treatment, where the gingiva cut line defines the boundary of the orthodontic appliance relative to the gingival margin and may be adjusted to improve retention, comfort, or aesthetics. The recommended updates may include adjusting interproximal reduction (IPR) for one or more teeth, such as modifying the amount, location, or staging of enamel reduction between adjacent teeth to create space for tooth movement. The recommended updates may include changing a pontic to a semi-pontic at a specified tooth position, where a pontic represents a replacement for a missing tooth in the appliance and a semi-pontic provides partial coverage of an edentulous space. These recommended updates may be generated by the trained machine learning model in response to natural language comments from the treatment provider specifying desired changes to the dental treatment plan.

The AI model may be an LLM. The AI model may be configured to generate the recommended updates based on natural language input. In some embodiments, the one or more AI models may be configured to interpret the semantic meaning of the first comments and translate the natural language instructions into specific treatment planning operations. The AI models may utilize contextual understanding to determine the appropriate scope and magnitude of the recommended updates based on the terminology and phrasing used in the first comments. For example, when a treatment provider indicates a desire to “speed up treatment” or “reduce the number of stages,” the AI model may determine specific adjustments to staging parameters, tooth movement rates, or appliance configurations that would achieve the stated objective. The AI models may further be configured to identify ambiguous or incomplete instructions in the first comments and generate clarifying questions or provide multiple alternative interpretations for user selection. In some embodiments, the AI models may access stored doctor preferences, historical treatment plan data, or clinical guidelines via retrieval-augmented generation techniques to inform the generation of recommended updates that align with the treatment provider's established practices and preferences. The processing of the first comments may include tokenization of the natural language input, extraction of key treatment parameters and objectives, mapping of the extracted information to available treatment planning operations, and generation of a structured set of recommended updates that can be presented to a user for acceptance or modification.

The recommended updates may include any adjustments to the treatment plan. The adjustments may be changing an order of various treatment steps, adjusting how many steps are performed, adjusting final outcomes or any intervening operations, etc. Some examples included changing a number of stages (e.g., a number of appliances) included in the treatment plan, adjusting orthopedic attachments, adjusting a degree of correction (e.g., adding over-correction for one or more teeth), adjusting torque applied during treatment to one or more teeth, adjusting extrusion or intrusion of one or more teeth, or adjusting a gingiva cut line of an appliance used in treatment.

At block 414, process logic generates an updated dental treatment plan including the one or more recommended updates, responsive to obtaining user acceptance of the one or more recommended updates. For example, the updates may be provided as recommended, pre-filled, or default actions in a treatment planning software, and a technician may accept these actions to provide user acceptance of the updates. The user acceptance may be obtained through various mechanisms, including selection of an accept button within a graphical user interface, confirmation through a chat-based interface, approval of recommended changes displayed in a review panel, or acceptance of suggested keyboard shortcut sequences that implement the recommended updates. In some embodiments, the user acceptance may be implicit, such as when a technician proceeds with treatment plan finalization without modifying the recommended updates. Generating the updated dental treatment plan may include communicating the treatment plan to a practitioner, e.g., by providing the treatment plan to a doctor device. The communication may include transmitting the updated dental treatment plan via network 130 to treatment provider device 120, where the updated plan may be displayed via graphical user interface 124 for practitioner review and final approval.

In some embodiments, generating an updated dental treatment plan may not be based on providing actions in a treatment planning software. For example, generating the updated dental treatment plan may be based on directly updating treatment operations (e.g., adjusting one or more plans for fabrication of treatment appliances), updating predictions of treatment outcomes (e.g., updating a three-dimensional model of final tooth positions for an orthodontic treatment), or the like. In some embodiments, an “AI technician” may work directly on a three-dimensional model/mesh of a dental arch. Generating the updated dental treatment plan may include predicting resulting treatment outcomes (e.g., three-dimensional mesh) based on comments input by one or more practitioners. In such embodiments, the AI model may receive the practitioner comments along with a current three-dimensional representation of the patient's dentition and directly compute modifications to the mesh geometry that implement the requested changes. The AI model may adjust vertex positions, modify surface normals, or alter mesh topology to reflect the updated tooth positions, orientations, and arrangements specified by the practitioner comments. In some embodiments, such implementations may in essence bypass treatment planning software. In some embodiments, the AI model(s) may be trained or configured to map practitioner comments to actions in the three-dimensional mesh space. Training the AI model for direct mesh manipulation may include providing training data comprising pairs of practitioner comments and corresponding mesh transformations, where the mesh transformations represent the geometric changes that implement the treatment modifications described in the comments. The trained AI model may learn to predict appropriate mesh deformations, vertex displacements, and surface modifications based on natural language descriptions of desired treatment outcomes.

Method 400B may include further actions. In some embodiments, the dental treatment plan may include or enable the generation of data for manufacturing one or more appliances for performing the treatment of the dental treatment plan. For example, one or more appliances may be used to adjust tooth positions, and manufacturing data for fabricating the appliances may be generated as part of the dental treatment plan or based on the dental treatment plan. The manufacturing data may include specifications for mold geometries, appliance shell thicknesses, cutline positions, attachment locations, and other parameters for appliance fabrication. The manufacturing data may be formatted as digital models suitable for input to rapid prototyping machines, such as stereolithography printers or other additive manufacturing systems. Manufacturing operations may be initiated by the process logic, e.g., based on the manufacturing data to manufacture appliances for enacting the treatment plan. Initiating manufacturing operations may include transmitting the manufacturing data to a fabrication facility, queuing the manufacturing job in a production scheduling system, or directly controlling fabrication equipment to begin producing the appliances. In some embodiments, the process logic may monitor the manufacturing operations and provide status updates to the treatment provider regarding appliance production progress. The manufactured appliances may then be shipped to the treatment provider for application to the patient's teeth in accordance with the updated dental treatment plan.

FIG. 4C is a flow diagram of a method 400C for training a model (e.g., a machine learning model, an AI model) to perform treatment plan updates based on natural language input, according to some embodiments. At block 420, process logic obtains first training data including natural language comments corresponding to target updates to a dental treatment plan. The natural language comments may be provided via one or more of a number of different input methods, including input text field, chat function, email or other free form input, spoken input which may be processed (e.g., via AI) into text, or the like. The dental treatment plan may be or include an orthodontic procedure to correct tooth misalignment, malocclusion, or other dental or orthodontic disorders.

At block 422, process logic obtains second training data including treatment planning software actions corresponding to the natural language comments. In some embodiments, the first and/or second training data may be based on user feedback, e.g., users may provide input to the system based on whether previous predictions of correlations between natural language comments and treatment planning updates were accurate, and the model may be trained based on the accuracy of previous predictions.

At block 424, process logic trains a machine learning model to predict treatment planning software actions based on natural language comments. The training is performed by providing the first training data as training input and corresponding second training data as target output.

Performing training operations of the machine learning model may be done in a number of ways. For example, parameters of a pre-existing LLM may be adjusted to form a custom-tuned LLM for performing operations in association with updating treatment plans. In some embodiments, an LLM or natural language processing (NLP) model may be trained from scratch for the purpose of generating and updating treatment plans. In some embodiments, adjusting parameters of the LLM may include performing parameter-efficient fine tuning methods.

FIG. 4D is a flow diagram of a method 400D for generating treatment plan updates based on output of a trained machine learning model, according to some embodiments. The method 400D illustrates a process by which natural language comments from a treatment provider can be transformed into actionable updates to a dental treatment plan through the application of trained machine learning models. At block 430, process logic obtains first comments associated with first target updates to a dental treatment plan or instructions for a dental treatment plan. The first comments may be obtained through various input mechanisms, including text entry fields within a graphical user interface, chat-based interfaces, email communications, or voice input that has been processed through speech-to-text conversion. The dental treatment plan may be or include an orthodontic procedure to correct at least one of malocclusion or tooth misalignment, and/or to correct other dental issues (e.g., caries, gingival recession, narrow palate, etc.). With regards to an orthodontic treatment plan, the dental treatment plan may specify a sequence of treatment stages, each stage corresponding to an incremental repositioning of one or more teeth toward a target arrangement. The first comments may indicate desired modifications to and/or inclusions for the treatment plan, such as changes to the number of treatment stages, adjustments to tooth movement parameters, modifications to attachment configurations, alterations to interproximal reduction specifications, or other treatment plan parameters.

At block 432, process logic provides the first comments to a trained machine learning model. The first comments may include natural language input provided by a doctor or practitioner. The trained machine learning model may be configured to generate treatment data based on natural language input. The trained machine learning model may be a large language model that has been fine-tuned or configured through prompt engineering to interpret dental treatment terminology and generate appropriate treatment plan modifications. In some embodiments, the trained machine learning model may access stored doctor preferences through retrieval-augmented generation techniques, enabling the model to generate updates that align with the treatment provider's established practices and preferences. The trained machine learning model may be configured to interpret the semantic meaning of the first comments and translate the natural language instructions into specific treatment planning operations or modifications to treatment plan parameters. In some embodiments, the trained machine learning model may generate clarifying questions or provide multiple alternative interpretations when the first comments are ambiguous or incomplete.

In some embodiments, two technical implementation approaches may be utilized for generating the first output from the trained machine learning model. A first approach, referred to as a hybrid approach, involves the trained machine learning model outputting commands for treatment planning software. In the hybrid approach, the treatment planning software complies with medical regulations, and all possible actions of the trained machine learning model are within the approved scope of the treatment planning software. The hybrid approach may provide better understandability, as the trained machine learning model behaves similarly to a human technician working with the treatment planning software. In the hybrid approach, the trained machine learning model maps the first comments to function calls or operations within the treatment planning software, such as adjusting staging, modifying interproximal reduction parameters, adding or removing attachments, applying three-dimensional controls, or updating gingiva representations. The treatment planning software then executes these operations to modify the three-dimensional model of patient dentition, rather than the trained machine learning model modifying the three-dimensional model directly. This approach ensures that medical constraints are maintained because the trained machine learning model only acts on the function calls of the treatment planning software.

A second approach, referred to as an end-to-end approach, involves the trained machine learning model directly modifying three-dimensional models of patient dentition. In the end-to-end approach, the intermediate treatment planning software is not required, which may result in greater efficiency. However, the end-to-end approach may be harder to develop and train, as the action space is significantly larger. The end-to-end approach may also raise additional regulatory considerations and may provide less understandability compared to the hybrid approach. In the end-to-end approach, the trained machine learning model may directly add or remove treatment stages, modify three-dimensional tooth positions and orientations, adjust attachment placements, and make other modifications to any part of the treatment plan. The end-to-end approach may enable a doctor to interact with a text interface and immediately receive updated treatment plan results, such as requesting an additional attachment on a specific tooth and seeing the treatment plan update in real time without involvement of a technician or intermediate treatment planning software.

At block 434, process logic obtains from the trained machine learning model first output including a first updated dental treatment plan. The first output may include machine-readable instructions specifying modifications to the dental treatment plan, treatment planning software actions to implement the requested changes, or a complete updated treatment plan incorporating the modifications indicated by the first comments. In the hybrid approach, the first output may comprise commands for the treatment planning software, such as keyboard shortcut sequences or function calls to an application programming interface of the treatment planning software, which are then executed by the treatment planning software to generate the first updated dental treatment plan. In the end-to-end approach, the first output may comprise a three-dimensional model directly modified by the trained machine learning model based on the first comments. In some embodiments, multiple rounds of updates may be performed, including second comments to adjust the updated plan, second output of the model, second updated dental treatment plan, etc. This iterative process enables treatment providers to refine the treatment plan through successive natural language interactions with the trained machine learning model. The first updated dental treatment plan may be or include predictions of patient dentition after completion of the dental treatment plan, e.g., a three-dimensional model predicting the location and orientation of the patient's teeth after treatment is completed. The three-dimensional model may represent the expected final tooth positions, orientations, and arrangements resulting from execution of the updated treatment plan. In some embodiments, the first output may include intermediate treatment stages showing the predicted progression of tooth movement throughout the treatment process.

At block 436, process logic provides the first updated dental treatment plan to a user. Providing the first updated dental treatment plan to a user may include providing the first dental treatment plan to a computing device, e.g., a practitioner or treatment provider device. The first updated dental treatment plan may be transmitted via network 130 to treatment provider device 120, where the updated plan may be displayed via graphical user interface 124 for practitioner review and approval. The graphical user interface may present the updated treatment plan in a format that enables the treatment provider to visualize the proposed changes, compare the updated plan with the original plan, and evaluate the predicted treatment outcomes. In some embodiments, the graphical user interface may include interactive three-dimensional viewers that enable the treatment provider to manipulate views of the predicted dentition, including panning, rotating, and zooming operations to inspect the treatment results from various perspectives. In some embodiments, manufacturing data for one or more appliances associated with the treatment plan may be generated, manufacturing of the appliances may be initiated, the treatment plan may be stored in memory for later use, review, or retrieval, or the like. The manufacturing data may include specifications for mold geometries, appliance shell configurations, attachment locations, cutline positions, and/or other parameters necessary for fabrication of orthodontic appliances. Initiating manufacturing operations may include transmitting the manufacturing data to a fabrication facility, queuing the manufacturing job in a production scheduling system, or directly controlling fabrication equipment to begin producing the appliances in accordance with the updated treatment plan.

FIG. 4E is a flow diagram of a method 400E for updating a treatment protocol based on output of a trained machine learning model, according to some embodiments. At block 440, process logic obtains first input including a description of a dental treatment plan associated with a first target dental disorder.

At block 442, process logic provides the first input to a trained machine learning model. In some embodiments, a prompt is provided to a user (e.g., via a GUI) to obtain a number of descriptions of dental treatment plans. Each of the dental treatment plans may be associated with a different disorder. In some embodiments, natural language input associated with multiple (e.g., all) of the presented disorders may be collected, and protocols generated for each. In some cases, multiple rounds of input may be provided, e.g., based on refining, fine-tuning, or updating previously provided input, previously generated protocols, protocols that failed to generate correctly, or the like. In some embodiments, a default treatment protocol associated with the first target dental disorder may be presented to the treatment provider, and the first input may be obtained as a modification to the default treatment protocol. This approach may enable treatment providers to start from established baseline protocols and customize them according to their preferences.

At block 444, process logic obtains from the trained machine learning model a first treatment protocol for treating the first target dental disorder in association with the first input in a machine-readable format (e.g., machine-readable instructions). In some embodiments, the machine-readable format may comprise a domain-specific language (DSL) script expressing the first treatment protocol. The DSL script may be configured to be processed by a treatment planning engine to automatically generate treatment plans in accordance with the treatment protocol.

At block 446, process logic presents the first treatment protocol for user approval. The protocol may be presented in a target format, e.g., a format familiar to a treatment provider, a format easily understood by a treatment provider, etc. In some embodiments, presenting the protocol for user approval may include generating a medical algorithm based on the treatment protocol, and presenting the medical algorithm to a user (e.g., treatment provider) via a GUI. The medical algorithm may provide a visual representation of the treatment protocol steps and decision points in a format commonly used in clinical practice. In some embodiments, presenting the first treatment protocol for user approval may include presenting one or more test cases in association with the protocol. The test cases may include example dentition (e.g., model dentition for the target disorder). The test cases may include previous patient dentition. The protocol may be applied to the one or more test cases, and results of applying the protocol to the test cases may be presented to the user (e.g., treatment provider) for validation. In some embodiments, the first treatment protocol may be provided to a second dental treatment provider for use in treating patients of the second dental treatment provider, enabling sharing of protocols among practitioners.

At block 448, responsive to obtaining user approval, process logic stores the first treatment protocol in non-transitory memory.

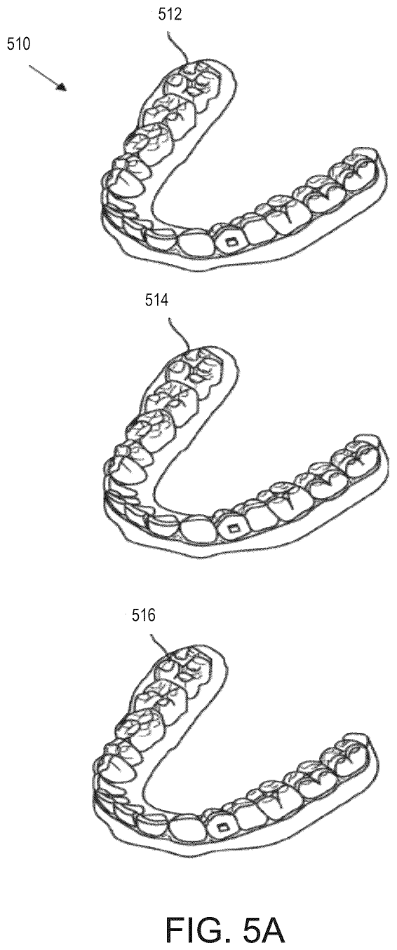

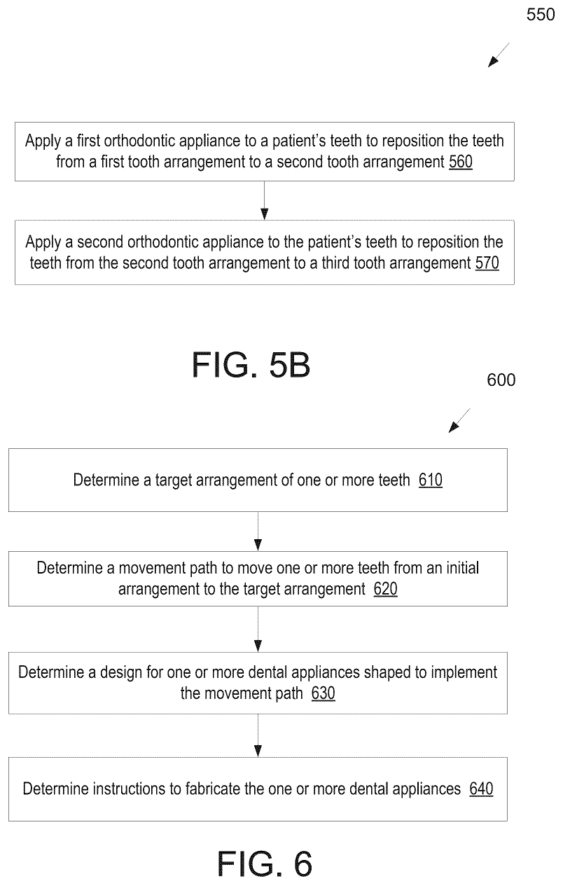

FIG. 5A illustrates a tooth repositioning system 510 including a plurality of appliances 512, 514, 516. The appliances 512, 514, 516 can be designed based on generation of a sequence of 3D models of dental arches, which may be generated according to the techniques discussed herein above. For example, treatment plans may be generated that include a sequence of 3D models. The treatment plans may be generated based on practitioner preferences, as codified in a set of treatment protocols generated based on natural language inputs in accordance with aspects of the present disclosure. The treatment plans may be adjusted or fine-tuned based on practitioner comments, which may be provided in natural language and either the treatment plans adjusted based on AI (e.g., LLM) parsing of the natural language input, or treatment planning software actions taken or recommended based on AI output from the natural language input.

Any of the appliances described herein can be designed and/or provided as part of a set of a plurality of appliances used in a tooth repositioning system, and may be designed in accordance with an orthodontic treatment plan generated in accordance with embodiments of the present disclosure. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 510 can include a first appliance 512 corresponding to an initial tooth arrangement, one or more intermediate appliances 514 corresponding to one or more intermediate arrangements, and a final appliance 516 corresponding to a target arrangement. A target tooth arrangement can be a planned final tooth arrangement selected for the patient's teeth at the end of all planned orthodontic treatment, as optionally output using a trained machine learning model. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 512, 514, 516 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

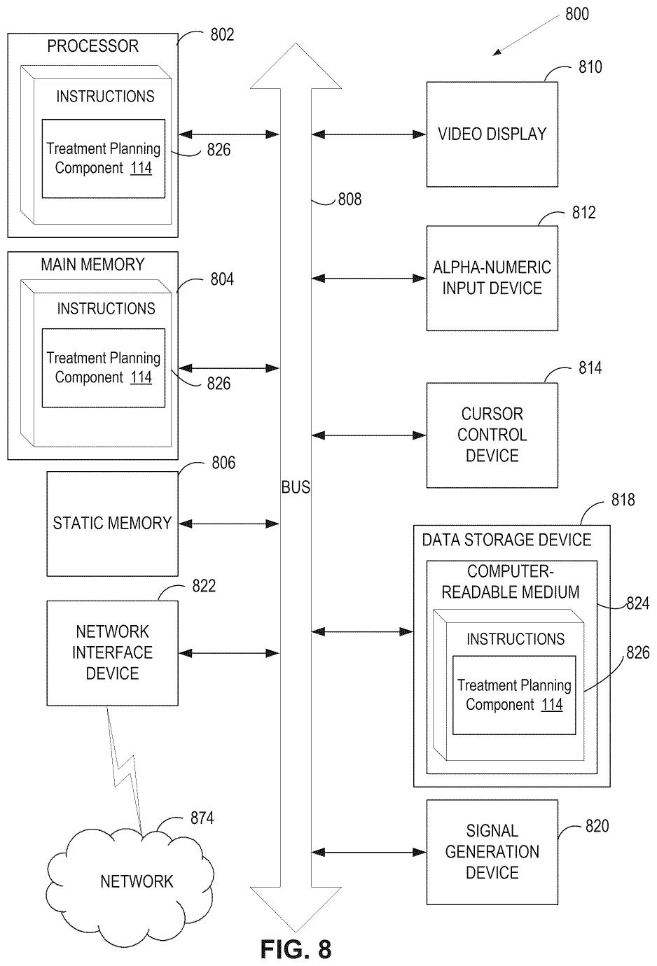

In an example of indirect fabrication, a mold of a patient's dental arch may be fabricated from a digital model of the dental arch generated by a trained machine learning model as described above, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 512, 514, 516 after the digital models of the appliances 512, 514, 516 have been processed by processing logic of a computing device, such as the computing device in FIG. 8. The processing logic may include hardware (e.g., circuitry, dedicated logic, programmable logic, microcode, etc.), software (e.g., instructions executed by a processing device), firmware, or a combination thereof. For example, one or more operations may be performed by a processing device executing dental treatment data generator 268 of FIG. 2.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a three dimensional (3D) virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images or scans to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner may determine a desired treatment outcome, which includes final positions and orientations for the patient's teeth. In one embodiment, dental treatment data generator 268 outputs a desired treatment outcome based on processing the virtual 3D model of the patient's dental arch (or other dental arch data associated with the virtual 3D model). Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. In one embodiment, for each treatment stage dental treatment data generator 268 outputs a different 3D model of the dental arch. The shape of each virtual 3D model will be different. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

In some instances, stereolithography (SLA), also known as optical fabrication solid imaging, is used to fabricate an SLA mold. In SLA, the mold is fabricated by successively printing thin layers of a photo-curable material (e.g., a polymeric resin) on top of one another. A platform rests in a bath of a liquid photopolymer or resin just below a surface of the bath. A light source (e.g., an ultraviolet laser) traces a pattern over the platform, curing the photopolymer where the light source is directed, to form a first layer of the mold. The platform is lowered incrementally, and the light source traces a new pattern over the platform to form another layer of the mold at each increment. This process repeats until the mold is completely fabricated. Once all of the layers of the mold are formed, the mold may be cleaned and cured.

Materials such as a polyester, a co-polyester, a polycarbonate, a polycarbonate, a thermopolymeric polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermopolymeric elastomer (TPE), a thermopolymeric vulcanizate (TPV) elastomer, a polyurethane elastomer, a block copolymer elastomer, a polyolefin blend elastomer, a thermopolymeric co-polyester elastomer, a thermopolymeric polyamide elastomer, or combinations thereof, may be used to directly form the mold. The materials used for fabrication of the mold can be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 512, 514, 516 can be pressure formed or thermoformed over the molds. Each mold may be used to fabricate an appliance that will apply forces to the patient's teeth at a particular stage of the orthodontic treatment. The appliances 512, 514, 516 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

Additional information may be added to the appliance. The additional information may be any information that pertains to the appliance. Examples of such additional information includes a part number identifier, patient name, a patient identifier, a case number, a sequence identifier (e.g., indicating which appliance a particular liner is in a treatment sequence), a date of manufacture, a clinician name, a logo and so forth. For example, after determining there is a probable point of damage in a digital design of an appliance, an indicator may be inserted into the digital design of the appliance. The indicator may represent a recommended place to begin removing the polymeric appliance to prevent the point of damage from manifesting during removal in some embodiments. In embodiments, the additional information may be automatically added to a generated 3D model by dental treatment data generator 268 in generation of the 3D model.

After an appliance is formed over a mold for a treatment stage, the appliance is removed from the mold (e.g., automated removal of the appliance from the mold), and the appliance is subsequently trimmed along a cutline (also referred to as a trim line). The processing logic may determine a cutline for the appliance. In one embodiment, dental treatment data generator 268 outputs a cutline for an appliance associated with a 3D model output by the dental arch generator 268. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

FIG. 5B illustrates a method 550 of orthodontic treatment using a plurality of appliances, in accordance with embodiments. The method 550 can be practiced using any of the appliances or appliance sets described herein. The method 550 can be applied based on treatment plans developed in accordance with treatment protocols based on natural language input, in accordance with aspects of the present disclosure. The method 550 can be applied based on treatment plans that have been updated via AI recognition of natural language comments or fine-tuning of generated treatment plans.

In block 560, a first orthodontic appliance is applied to a patient's teeth in order to reposition the teeth from a first tooth arrangement to a second tooth arrangement. In block 570, a second orthodontic appliance is applied to the patient's teeth in order to reposition the teeth from the second tooth arrangement to a third tooth arrangement. The method 550 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 6 illustrates a method 600 for designing an orthodontic appliance to be produced by direct or indirect fabrication, in accordance with embodiments. The method 600 can be applied to any embodiment of the orthodontic appliances described herein, and may be performed using one or more trained machine learning models in embodiments. Some or all of the blocks of the method 600 can be performed by any suitable data processing system or device, e.g., one or more processors configured with suitable instructions. Method 600 may be based on treatment protocols generated from natural language instructions, in accordance with aspects of the present disclosure. Method 600 may be based on treatment plans generated from or updated based on AI output from natural language input including treatment provider comments to update the treatment plans.

At block 610 a target arrangement of one or more teeth of a patient may be determined. The target arrangement of the teeth (e.g., a desired and intended end result of orthodontic treatment) can be received from a clinician in the form of a prescription, can be calculated from basic orthodontic principles, can be extrapolated computationally from a clinical prescription, and/or can be generated by a trained machine learning model such as treatment dental treatment data generator 268 of FIG. 2. With a specification of the desired final positions of the teeth and a digital representation of the teeth themselves, the final position and surface geometry of each tooth can be specified to form a complete model of the tooth arrangement at the desired end of treatment.

In block 620, a movement path to move the one or more teeth from an initial arrangement to the target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. An initial arrangement may be estimated by projecting some measurement of the patient's teeth to a latent space, and obtaining from the latent space a representation of the initial arrangement. From the obtained data, a digital data set such as a 3D model of the patient's dental arch or arches can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, optionally including measured or extrapolated hidden surfaces and root structures, as well as surrounding bone and soft tissue.

Having both an initial position and a target position for each tooth, a movement path can be defined for the motion of each tooth. Determining the movement path for one or more teeth may include identifying a plurality of incremental arrangements of the one or more teeth to implement the movement path. In some embodiments, the movement path implements one or more force systems on the one or more teeth (e.g., as described below). In some embodiments, movement paths are determined by a trained machine learning model such as treatment plan generator 276. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In some embodiments, a force system to produce movement of the one or more teeth along the movement path is determined. In one embodiment, the force system is determined by a trained machine learning model. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be considered. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In block 630, a design for one or more dental appliances shaped to implement the movement path is determined. In one embodiment, the one or more dental appliances are shaped to move the one or more teeth toward corresponding incremental arrangements. In some embodiments, results of one or more stages of treatment may be predicted by dental treatment data generator 268. Determination of the one or more dental or orthodontic appliances, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

In block 640, instructions for fabrication of the one or more dental appliances are determined or identified. In some embodiments, the instructions identify one or more geometries of the one or more dental appliances. In some embodiments, the instructions identify slices to make layers of the one or more dental appliances with a 3D printer. In some embodiments, the instructions identify one or more geometries of molds usable to indirectly fabricate the one or more dental appliances (e.g., by thermoforming plastic sheets over the 3D printed molds). The dental appliances may include one or more of aligners (e.g., orthodontic aligners), retainers, incremental palatal expanders, attachment templates, and so on.

In one embodiment, instructions for fabrication of the one or more dental appliances are generated by a trained model. In some embodiments, predictions of treatment progression and/or treatment appliances may be performed and/or aided by dental treatment data generator 268. The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by 3D printing a mold and thermoforming a plastic sheet over the mold.

Method 600 may comprise additional blocks: 1) The upper arch and palate of the patient is scanned intraorally to generate three dimensional data of the palate and upper arch; 2) The three dimensional shape profile of the appliance is determined to provide a gap and teeth engagement structures as described herein.

Although the above blocks show a method 600 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 600 may be performed with any suitable fabrication system or device, such as the embodiments described herein. Some of the blocks may be optional, and the order of the blocks can be varied as desired.

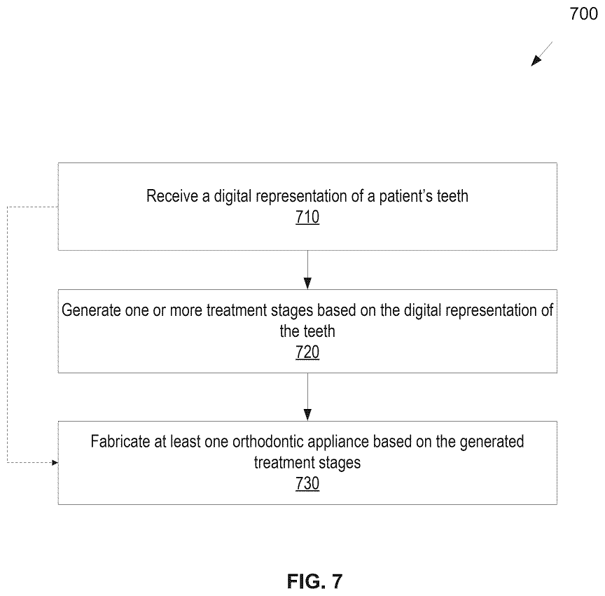

FIG. 7 illustrates a method 700 for digitally planning an orthodontic treatment and/or design or fabrication of an appliance, in accordance with embodiments. The method 700 can be applied to any of the treatment procedures described herein and can be performed by any suitable data processing system. The method 700 may be based on treatment protocols generated in accordance with natural language practitioner input, as described herein. The method 700 may be based on treatment plans updated based on natural language input, as described herein.

In block 710, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 720, one or more treatment stages are generated based on the digital representation of the teeth. In some embodiments, the one or more treatment stages are generated based on processing of input dental arch data by a trained machine learning model such as dental treatment data generator 268. Each treatment stage may include a generated 3D model of a dental arch at that treatment stage. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 730, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired. The fabrication of the appliance may include automated removal of the appliance from a mold (e.g., automated removal of an untrimmed shell from mold a using a shell removal device).

In some instances, staging of various arrangements or treatment stages may not be necessary for design and/or fabrication of an appliance. As illustrated by the dashed line in FIG. 7, design and/or fabrication of an orthodontic appliance, and perhaps a particular orthodontic treatment, may include use of a representation of the patient's teeth (e.g., receive a digital representation of the patient's teeth at block 710), followed by design and/or fabrication of an orthodontic appliance based on a representation of the patient's teeth in the arrangement represented by the received representation.

FIG. 8 is a block diagram illustrating a computer system 800, according to some embodiments. In some embodiments, computer system 800 may be connected (e.g., via a network, such as a Local Area Network (LAN), an intranet, an extranet, or the Internet) to other computer systems. Computer system 800 may operate in the capacity of a server or a client computer in a client-server environment, or as a peer computer in a peer-to-peer or distributed network environment. Computer system 800 may be provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 800 may include a processing device 802, a volatile memory 804 (e.g., Random Access Memory (RAM)), a non-volatile memory 806 (e.g., Read-Only Memory (ROM) or Electrically-Erasable Programmable ROM (EEPROM)), and a data storage device 818, which may communicate with each other via a bus 808.

Processing device 802 may be provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor.

Computer system 800 may further include a network interface device 822 (e.g., coupled to network 874). Computer system 800 also may include a video display unit 810 (e.g., an LCD), an alphanumeric input device 812 (e.g., a keyboard), a cursor control device 814 (e.g., a mouse), and a signal generation device 820.

In some embodiments, data storage device 818 may include a non-transitory computer-readable storage medium 824 (e.g., non-transitory machine-readable medium) on which may store instructions 826 encoding any one or more of the methods or functions described herein, including instructions encoding components of FIG. 1 (e.g., treatment planning component 114, graphical user interface 124, model 190, etc.) and for implementing methods described herein.

Instructions 826 may also reside, completely or partially, within volatile memory 804 and/or within processing device 802 during execution thereof by computer system 800, hence, volatile memory 804 and processing device 802 may also constitute machine-readable storage media.

While computer-readable storage medium 824 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “receiving,” “performing,” “providing,” “obtaining,” “causing,” “accessing,” “determining,” “adding,” “using,” “training,” “reducing,” “generating,” “correcting,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform methods described herein and/or each of their individual functions, routines, subroutines, or operations. Examples of the structure for a variety of these systems are set forth in the description above.

Example implementations are provided below.
A first example implementation comprises a method that includes obtaining first comments indicating changes to a dental treatment plan; processing the first comments using one or more artificial intelligence (AI) models, wherein the one or more AI models determine one or more recommended updates to the dental treatment plan that implement the changes from the first comments and output the one or more recommended updates to the dental treatment plan; and responsive to obtaining user acceptance of the one or more recommended updates, generating an updated dental treatment plan comprising the one or more recommended updates.
A second example implementation may extend the first example implementation. In the second example implementation, the method further comprises retrieving stored doctor preferences from a data store; and processing the stored doctor preferences together with the first comments using the one or more AI models, wherein the one or more recommended updates are generated in view of the stored doctor preferences.
A third example implementation may extend any of the first or second example implementations. In the third example implementation, the stored doctor preferences are generated based on at least one of: a questionnaire completed by a dental treatment provider, a chat-based interaction with a dental treatment provider, or historical treatment plan data associated with a dental treatment provider.
A fourth example implementation may extend any of the first through third example implementations. In the fourth example implementation, the stored doctor preferences are retrieved using a retrieval-augmented generation technique.
A fifth example implementation may extend any of the first through fourth example implementations. In the fifth example implementation, the method further comprises automatically generating the stored doctor preferences based on analysis of historical treatment plan modifications associated with a dental treatment provider.
A sixth example implementation may extend any of the first through fifth example implementations. In the sixth example implementation, obtaining the first comments comprises receiving voice input from a user and performing speech-to-text processing on the voice input to generate the first comments.
A seventh example implementation may extend any of the first through sixth example implementations. In the seventh example implementation, the first comments comprise natural language input, and the one or more AI models is configured to generate at least one of the one or more recommended updates or the updated dental treatment plan based on the natural language input.
An eighth example implementation may extend any of the first through seventh example implementations. In the eighth example implementation, the dental treatment plan is associated with treatment planning software, and output of the one or more AI models comprises instructions for operations performed within the treatment planning software.
A ninth example implementation may extend any of the first through eighth example implementations. In the ninth example implementation, the instructions for operations performed within the treatment planning software comprise one or more keyboard shortcut sequences corresponding to the one or more recommended updates.
A tenth example implementation may extend any of the first through ninth example implementations. In the tenth example implementation, the instructions for operations performed within the treatment planning software comprise one or more function calls to an application programming interface of the treatment planning software.
An eleventh example implementation may extend any of the first through tenth example implementations. In the eleventh example implementation, the method further comprises obtaining second comments indicating second changes to the dental treatment plan; processing the second comments using the one or more AI models, wherein the one or more AI models determine one or more second recommended updates to the dental treatment plan that implement the second changes from the second comments and output the one or more second recommended updates to the dental treatment plan; and responsive to obtaining user denial of the one or more second recommended updates, providing a prompt to a user to provide input comprising the first comments.
A twelfth example implementation may extend any of the first through eleventh example implementations. In the twelfth example implementation, the dental treatment plan comprises an orthodontic treatment plan to correct at least one of a malocclusion or tooth misalignment of a patient.
A thirteenth example implementation may extend any of the first through twelfth example implementations. In the thirteenth example implementation, the method further comprises generating manufacturing data of one or more dental appliances responsive to the updated dental treatment plan.
A fourteenth example implementation may extend any of the first through thirteenth example implementations. In the fourteenth example implementation, the method further comprises initiating manufacturing operations of the one or more dental appliances based on the manufacturing data.
A fifteenth example implementation may extend any of the first through fourteenth example implementations. In the fifteenth example implementation, the method further comprises providing the updated dental treatment plan to a computing device associated with a dental treatment provider.
A sixteenth example implementation may extend any of the first through fifteenth example implementations. In the sixteenth example implementation, the one or more AI models comprise a large language model (LLM).
A seventeenth example implementation may extend any of the first through sixteenth example implementations. In the seventeenth example implementation, the recommended updates comprise one or more of: reducing a number of stages included in the updated dental treatment plan; adjusting orthopedic attachments; adding over-correction for one or more teeth; adjusting torque applied during treatment to one or more teeth; adjusting extrusion or intrusion of one or more teeth; or adjusting gingiva cut line of an appliance used in treatment.
An eighteenth example implementation may extend any of the first through seventeenth example implementations. In the eighteenth example implementation, output of the one or more AI models is constrained to actions within a scope of operations approved for a treatment planning software.
A nineteenth example implementation may extend any of the first through eighteenth example implementations. In the nineteenth example implementation, the recommended updates comprise at least one of: adjusting interproximal reduction for one or more teeth; or changing a pontic to a semi-pontic at a specified tooth position.
A twentieth example implementation may extend any of the first through nineteenth example implementations. In the twentieth example implementation, the first comments are obtained via a chat-based interface, and the method further comprises providing immediate feedback to a user via the chat-based interface based on the one or more recommended updates.
A twenty-first example implementation may extend any of the first through twentieth example implementations. In the twenty-first example implementation, the method further comprises predicting additional comments by the one or more AI models based on the first comments, wherein the first comments are incomplete and the additional comments comprise a completed version of the first comments, and wherein the additional comments are processed together with the first comments to generate the one or more recommended updates.
A twenty-second example implementation may extend any of the first through twenty-first example implementations. In the twenty-second example implementation, the one or more recommended updates comprise a plurality of sequential operations that implement a change specified in the first comments requiring modification of multiple stages of the dental treatment plan.
A twenty-third example implementation may extend any of the first through twenty-second example implementations. In the twenty-third example implementation, the method further comprises automatically executing the one or more recommended updates within a treatment planning software under supervision of a dental technician.
A twenty-fourth example implementation may extend any of the first through twenty-third example implementations. In the twenty-fourth example implementation, the recommended updates comprise at least one of: applying three-dimensional controls to the dental treatment plan; adjusting staging for one or more teeth; updating a gingiva representation; or adding one or more attachments.
A twenty-fifth example implementation comprises a method that includes obtaining first training data comprising natural language comments corresponding to target updates of a dental treatment plan; obtaining second training data comprising treatment planning software actions corresponding to the natural language comments; and training a machine learning model to predict treatment planning software actions based on natural language comments by providing the first training data as training input and the second training data as target output.
A twenty-sixth example implementation may extend the twenty-fifth example implementation. In the twenty-sixth example implementation, training the machine learning model comprises adjusting one or more parameters of a large language model.
A twenty-seventh example implementation may extend any of the twenty-fifth or twenty-sixth example implementations. In the twenty-seventh example implementation, the dental treatment plan comprises an orthodontic procedure to correct at least one of a malocclusion or tooth misalignment.
A twenty-eighth example implementation may extend any of the twenty-fifth through twenty-seventh example implementations. In the twenty-eighth example implementation, the method further comprises generating the first training data and the second training data by generating predicted treatment planning software actions, and obtaining user feedback in connection with user-evaluated accuracy of the predicted treatment planning software actions.
A twenty-ninth example implementation may extend any of the twenty-fifth through twenty-eighth example implementations. In the twenty-ninth example implementation, the method further comprises generating the first training data based on technician acceptance or rejection of predicted treatment planning software actions output by the machine learning model.
A thirtieth example implementation comprises a method that includes obtaining first comments associated with first target updates to a dental treatment plan; providing the first comments to a trained machine learning model; obtaining from the trained machine learning model first output comprising a first updated dental treatment plan; and providing the first updated dental treatment plan to a computing device associated with a dental treatment provider.
A thirty-first example implementation may extend the thirtieth example implementation. In the thirty-first example implementation, the first updated dental treatment plan comprises a three-dimensional model of predicted patient dentition upon execution of the first updated dental treatment plan.
A thirty-second example implementation may extend any of the thirtieth or thirty-first example implementations. In the thirty-second example implementation, the first comments comprise a natural language input, and the trained machine learning model is configured to generate the first updated dental treatment plan based on natural language input.
A thirty-third example implementation may extend any of the thirtieth through thirty-second example implementations. In the thirty-third example implementation, the method further comprises obtaining second comments associated with second target updates to the first updated dental treatment plan; providing the second comments to the trained machine learning model; and obtaining from the trained machine learning model a second output comprising a second updated dental treatment plan.
A thirty-fourth example implementation may extend any of the thirtieth through thirty-third example implementations. In the thirty-fourth example implementation, the first updated dental treatment plan comprises an orthodontic procedure to correct at least one of a malocclusion or tooth misalignment.
A thirty-fifth example implementation may extend any of the thirtieth through thirty-fourth example implementations. In the thirty-fifth example implementation, the method further comprises generating manufacturing data of one or more dental appliances responsive to the first updated dental treatment plan.
A thirty-sixth example implementation may extend any of the thirtieth through thirty-fifth example implementations. In the thirty-sixth example implementation, the method further comprises initiating manufacturing operations of the one or more dental appliances based on the manufacturing data.
A thirty-seventh example implementation may extend any of the thirtieth through thirty-sixth example implementations. In the thirty-seventh example implementation, the first updated dental treatment plan comprises a three-dimensional model directly modified by the trained machine learning model based on the first comments.
A thirty-eighth example implementation comprises a method that includes obtaining, by a processing device, first input comprising a description of a dental treatment protocol associated with a first target dental disorder; providing the first input to a trained machine learning model; obtaining from the trained machine learning model a first treatment protocol for treating the first target dental disorder in association with the first input in a machine readable format; presenting the first treatment protocol for user approval; and responsive to obtaining user approval, storing the first treatment protocol in non-transitory memory.
A thirty-ninth example implementation may extend the thirty-eighth example implementation. In the thirty-ninth example implementation, the machine readable format comprises a domain-specific language script expressing the first treatment protocol.
A fortieth example implementation may extend any of the thirty-eighth or thirty-ninth example implementations. In the fortieth example implementation, the method further comprises providing the first treatment protocol to a second dental treatment provider for use in treating patients of the second dental treatment provider.
A forty-first example implementation may extend any of the thirty-eighth through fortieth example implementations. In the forty-first example implementation, the method further comprises presenting a default treatment protocol associated with the first target dental disorder; and obtaining the first input as a modification to the default treatment protocol.
A forty-second example implementation may extend any of the thirty-eighth through forty-first example implementations. In the forty-second example implementation, presenting the first treatment protocol for user approval comprises generating a medical algorithm based on the treatment protocol in the machine readable format; and presenting the medical algorithm via a graphical user interface.
A forty-third example implementation may extend any of the thirty-eighth through forty-second example implementations. In the forty-third example implementation, the method further comprises providing a prompt for a user to input a plurality of descriptions of dental treatment plans associated with a plurality of dental disorders, comprising the first target dental disorder; obtaining second input corresponding to the prompt in association with a second target dental disorder; obtaining from the trained machine learning model a second treatment protocol corresponding to the second target dental disorder; and presenting the second treatment protocol for user approval.
A forty-fourth example implementation may extend any of the thirty-eighth through forty-third example implementations. In the forty-fourth example implementation, presenting the first treatment protocol for user approval comprises presenting one or more test cases, comprising example dentition or previous patient dentition; applying the first treatment protocol to the one or more test cases; generating a representation of results of the first treatment protocol on dentition of the one or more test cases; and presenting the representation for a graphical user interface.
A forty-fifth example implementation may extend any of the thirty-eighth through forty-fourth example implementations. In the forty-fifth example implementation, the method further comprises obtaining second input comprising an initial description of the dental treatment plan; providing the second input to the trained machine learning model; obtaining from the trained machine learning model a second treatment protocol in association with the second input in a machine-readable format; and presenting the second treatment protocol for user approval, wherein the first input is in response to user review of the second treatment protocol.
A forty-sixth example implementation may extend any of the first through forty-fifth example implementations. In the forty-sixth example implementation, a computer readable storage medium comprises instructions that, when executed by a processing device, cause the processing device to perform the method of any of the first through forty-fifth example implementations.
A forty-seventh example implementation may extend any of the first through forty-fifth example implementations. In the forty-seventh example implementation, a system comprises: a memory; and one or more processors configured to execute instructions from the memory to perform the method of any of the first through forty-fifth example implementations.

The above description is intended to be illustrative, and not restrictive. Although the present disclosure has been described with references to specific illustrative examples and embodiments, it will be recognized that the present disclosure is not limited to the examples and embodiments described. The scope of the disclosure should be determined with reference to the following claims, along with the full scope of equivalents to which the claims are entitled.

1. A method, comprising:
obtaining first comments indicating changes to a dental treatment plan;
processing the first comments using one or more artificial intelligence (AI) models, wherein the one or more AI models determine one or more recommended updates to the dental treatment plan that implement the changes from the first comments and output the one or more recommended updates to the dental treatment plan; and
responsive to obtaining user acceptance of the one or more recommended updates, generating an updated dental treatment plan comprising the one or more recommended updates.

2-6. (canceled)

7. The method of claim 1, wherein the first comments comprise natural language input, and wherein the one or more AI models is configured to generate at least one of the one or more recommended updates or the updated dental treatment plan based on the natural language input.

8. The method of claim 1, wherein:
the first comments are obtained by a technician device from a practitioner device;
the dental treatment plan is associated with treatment planning software; and
output of the one or more AI models comprises instructions for operations performed within the treatment planning software by the technician device.

9. (canceled)

10. The method of claim 8, wherein the instructions for operations performed within the treatment planning software comprise one or more function calls to an application programming interface of the treatment planning software.

11. The method of claim 1, further comprising:
obtaining second comments indicating second changes to the dental treatment plan;
processing the second comments using the one or more AI models, wherein the one or more AI models determine one or more second recommended updates to the dental treatment plan that implement the second changes from the second comments and output the one or more second recommended updates to the dental treatment plan; and
responsive to obtaining user denial of the one or more second recommended updates, providing a prompt to a user to provide input comprising the first comments.

12-15. (canceled)

16. The method of claim 1, wherein the one or more AI models comprise a large language model (LLM).

17. The method of claim 1, wherein the recommended updates comprise one or more of:
reducing a number of stages included in the updated dental treatment plan;
adjusting orthopedic attachments;
adding over-correction for one or more teeth;
adjusting torque applied during treatment to one or more teeth;
adjusting extrusion or intrusion of one or more teeth; or
adjusting gingiva cut line of an appliance used in treatment.

18. The method of claim 1, wherein output of the one or more AI models is constrained to actions within a scope of operations approved for a treatment planning software.

19-22. (canceled)

23. The method of claim 1, further comprising:
automatically executing the one or more recommended updates within a treatment planning software under supervision of a dental technician.

24. (canceled)

25. A computer readable storage medium comprises instructions that, when executed by a processing device, cause the processing device to perform operations comprising:
obtaining first training data comprising natural language comments corresponding to target updates of a dental treatment plan;
obtaining second training data comprising treatment planning software actions corresponding to the natural language comments; and
training a machine learning model to predict treatment planning software actions based on natural language comments by providing the first training data as training input and the second training data as target output.

26. The computer readable storage medium of claim 25, wherein training the machine learning model comprises adjusting one or more parameters of a large language model.

27. The computer readable storage medium of claim 25, wherein the dental treatment plan comprises an orthodontic procedure to correct at least one of a malocclusion or tooth misalignment.

28. The computer readable storage medium of claim 25, wherein the operations further comprise generating the first training data and the second training data by generating predicted treatment planning software actions, and obtaining user feedback in connection with user-evaluated accuracy of the predicted treatment planning software actions.

29. The computer readable storage medium of claim 25, wherein the operations further comprise generating the second training data based on technician acceptance or rejection on a technician device of predicted treatment planning software actions output by the machine learning model, wherein the natural language comments are generated using a practitioner device.

30. A system, comprising a memory and one or more processors configured to execute instructions from the memory to:
obtain first comments associated with first target updates to a dental treatment plan;
provide the first comments to a trained machine learning model;
obtain from the trained machine learning model first output comprising a first updated dental treatment plan; and
provide the first updated dental treatment plan to a computing device associated with a dental treatment provider.

31. (canceled)

32. The system of claim 30, wherein the first comments comprise a natural language input, and wherein the trained machine learning model is configured to generate the first updated dental treatment plan based on natural language input.

33. The system of claim 30, wherein the one or more processors are further to:
obtain second comments associated with second target updates to the first updated dental treatment plan;
provide the second comments to the trained machine learning model; and
obtain from the trained machine learning model a second output comprising a second updated dental treatment plan.

34. The system of claim 30, wherein the first updated dental treatment plan comprises an orthodontic procedure to correct at least one of a malocclusion or tooth misalignment.

35. The system of claim 30, wherein the one or more processors are further to generate manufacturing data of one or more dental appliances responsive to the first updated dental treatment plan.

36. (canceled)

37. The system of claim 30, wherein the first updated dental treatment plan comprises a three-dimensional model directly modified by the trained machine learning model based on the first comments.

38-47. (canceled)
